(19) United States
(12) Patent Application Publication
(10) Pub. No.: US 2025/0127881 A1
(43) Pub. Date: Apr. 24, 2025

(54) An Immunogenic Composition of Disease-Associated Antigens for Use in a Vaccine, Antibody Production and Immunodiagnostic Tests

(71) Applicant: Fikret Sahin, Ankara (TR)

(72) Inventor: Fikret Sahin, Ankara (TR)

(21) Appl. No.: 18/835,091

(22) PCT Filed: Feb. 1, 2022
(86) PCT No.: PCT/TR2022/050090

§ 371 Date: Aug. 1, 2024

(51) Int. Cl. A61K 39/215; A61K 39/00; A61K 39/385; A61P 31/14; C07K 14/005; C07K 16/10; C12N 15/86

(52) U.S. Cl. CPC A61K 39/215; A61K 39/385; A61P 31/14; C07K 14/005; C07K 16/1003; C12N 15/86; A61K 2039/545; A61K 2039/575; A61K 2039/6075; C12N 2740/10043; C12N 2770/20022; C12N 2770/20034; C12N 2830/003

(57) ABSTRACT

The invention is based on the deletion of 21 amino acids from the C-terminal region of the S2 subunit of SARS-CoV2-S protein and transporting the Si subunit, which is fused to S2 to the cell membranes. In this way, the presentation of the antigenic S1 subunit of SARS-CoV2-S protein in large amounts and in its natural structure in the cell membrane and its use as a whole cell or cell membrane has been determined as a new vaccination protocol for the SARS-CoV-2. Designing the S2 subunit of SARS-CoV2-S protein as a carrier, fusing any bacterial, viral, and tumor proteins with antigenic properties and transporting it to the cell membrane will be a comprehensive vaccination protocol that will cover all bacteria, viruses and even tumors.

This invention is about a vaccination method developed for all viruses, especially Covid-19, bacteria, and tumor associated antigens in a short time, in large quantities, safe and effective.

Preparation of cell membrane presenting covid-19 spike protein in its structure, use for vaccination, antibody production and preparation of ELISA for antibody detection for covid-19.

### BACKGROUND

The Covid-19 pandemic, caused by the novel coronavirus SARS-CoV-2, has caused massive global chaos. The most important hope for reducing the risk of disease and infection is considered to be the development of an effective and in large quantities SARS-CoV2 vaccines method.

The S protein, the most crucial surface protein of SARS-CoV-2, is a large trimeric transmembrane glycoprotein with many glycosylation modifications that form a unique corolla structure on the surface of the virus. The S protein consists of the S1 and S2 subunits. The total length of SARS-CoV2-S is 1273 amino acids and consists of a signal peptide (amino acids 1-13) located at the N-terminus, the S1 subunit (14-685 residues), and the S2 subunit (686-1273 residues); the last two regions are responsible for receptor binding and membrane fusion, respectively. In the S1 subunit, there is a N-terminal domain (14-305 residues) and a receptor-binding domain (319-541 residues); the fusion peptide (FP) (788-806 residues), heptapeptide repeat sequence 1 (HR1) (912-984 residues), HR2 (1163-1213 residues), TM domain (1213-1237 residues), and cytoplasm domain (1237-1273 residues) comprise the S2 subunit (Huang, et all, 2020). The S1 subunit mainly contains the receptor-binding domain (RBD), which is responsible for recognizing receptors. Mutations involving RBD-associated amino acids lead to changes in the preference and infection characteristics of viral species. The S2 subunit comprises elements that are essential for membrane fusion. The S protein plays a vital role in the binding of the virus to, and its fusion with, the host cell membrane receptor. Given its crucial role in SARS-CoV2 infection and adaptive immunity, the S protein is an important target site for neutralizing antibodies and a key target for vaccine design.

Roughly, 3 years after the first case of COVID-19 was identified, a number of vaccines received emergency use authorization (EUA) by the US FDA. The novel technologies used to develop these vaccines also contributed to the speed with which candidate vaccines were developed.

Among the 64 COVID-19 vaccines in clinical trials, 44 are based on the S protein, of which 29 are based on the full-length S protein (65.91%, 29/44), 14 on the S protein RBD (31.82%, 14/44), and 1 on the S protein S-2P (2.27%, 1/44). Furthermore, both the Moderna mRNA-1273 vaccine and Pfizer-BioNtech BNT162b2 vaccines were designed based on the nucleotide sequence of the S protein (Golob et al. 2021).

Three major whole inactivated virus (WIV) vaccines are beginning to be distributed widely. Several protein-based and protein particle-based vaccines are advancing with promising results (Hotez and Bottazzi, 2021)

Vaccines have traditionally taken more than 10 years from identification of targets and selection of platforms to completion of phase I, II, and III clinical trials and ultimate regulatory approval. The massive scale of SARS-CoV2 infection worldwide and the enormous death toll called for a different approach. In May 2020, the US government established Operation Warp Speed, a public-private partnership to accelerate the development, production, and distribution of COVID-19 vaccines, therapeutics, and diagnostics (Golob, et Al. 2021).

Despite the urgency, development of SARS-CoV2 vaccines proceeded through the same steps as other vaccines: target identification, platform selection, design of candidate vaccines, and phased human clinical trials. In phase I and II clinical trials, immunological response to SARS-CoV2 vaccine is mainly determined based on detection of antibodies against the S protein and the RBD. Protection against SARS-CoV2 infection is assumed based on detection of antibody titers similar to that found in convalescent plasma and results of antibody detection by ELISA comparable to levels that are effective in virus neutralization assays.

Phase I/II trials focus on dose finding, safety, and measures of immunogenicity (e.g., the development of neutralizing antibodies against SARS-CoV2-S protein). Phase III trials needed to be double blind, randomized, and placebo controlled, and a minimum of 2 months of safety data was required for consideration for emergency use authorization (EUA) (Golob, et Al. 2021).

Virus-specific antibodies (IgA and IgM followed by IgG) against viral surface glycoproteins, mainly the spike (S) glycoprotein, are detected within 7 to 10 days after illness onset. Studies suggest that neutralizing antibodies are short lived, falling to undetectable levels within a few months.

Currently reported efficacy estimates are based on data during the first 3-4 months after the first dose of candidate vaccines. Therefore, in order to stop the spread of the virus, at least 60% of the entire human population should be immunized within 2-3 months. However, no vaccine production method mentioned above can produce enough vaccinies to vaccinate such a large population in a short time. Therefore, ongoing challenges of meeting vaccination goals include production of enough vaccinies to vaccinate a large population and supply chain issues, timely and equitable distribution, and the constant threat of emerging virus variants.

Therefore, effective vaccine methods that can be produced in high quantities in a short time are needed.

Some studies indicate that the SARS-CoV2 spike proteins alone crosses the blood-brain barrier (Rhea, et Al., 2021).

In addition, researchers found that the S1 subunit of SARS-CoV2 S protein, without the whole virion or genome, triggered growth signals in cultured human blood vessels from the lungs and mediated cell signaling promotes the hyperplasia and/or hypertrophy of vascular smooth muscle and endothelial cells, contributing to the complex cardiovascular outcomes (Suzuki, et Al. 2020).

This invention is related to the method of producing safe and effective SARS-CoV2 vaccine in large quantities in a short time.

The invention is based on the intracellular target signal region of the spike protein of the Coronovirus which is described by Lontok et al. (Lontok et al, 2004).

According to their findings Coronavirus budding at the endoplasmic reticulum-Golgi intermediate compartment (ERGIC) requires accumulation of the viral envelope proteins at this point in the secretory pathway. They demonstrate that the spike (S) protein from the group 3 coronavirus infectious bronchitis virus (IBV) contains a canonical dilysine endoplasmic reticulum retrieval signal (—KKXX—COOH) in its cytoplasmic tail. This signal can retain a chimeric reporter protein in the ERGIC and when mutated allows transport of the full-length S protein as well as the chimera to the plasma membrane. Recently Boson et al. show that envelope (E), and membrane (M) proteins regulate intracellular trafficking of S as well as its intracellular processing. Indeed, the data reveal that S is relocalized at ERGIC or Golgi compartments upon coexpression of E or M, as observed in SARS-CoV-2-infected cells, which prevents syncytia formation. They also showed that a C-terminal retrieval motif in the cytoplasmic tail of S is required for its M-mediated retention in the ERGIC, whereas E induces S retention by modulating the cell secretory pathway. They also showed that the S protein with the 21 amino acid deletion they made in C terminal region was transported to the plasma membrane and basically located in the cell membrane, which cause syncytia formation.

They concluded that the C-terminal moiety of SARS-CoV2-S cytoplasmic tail is essential for retention of S protein in ERGIC compartment (Boson, et all. 2020).

Therefore, I hypothesized that when the SARS-CoV2-S Δ21 protein is expressed ectopically in the cell without M expression, it will be densly located in the cytoplasmic membrane without remaining in ERGIC. In addition, the absence of E protein will increase the secretion of SARS-CoV2-S towards the cell membrane and prevent SARS-CoV2-S retention in ERGIC. Importantly, SARS-CoV2-S Δ21 protein will be located in the cell membrane in its most natural form compared to the conditions of obtaining S protein by other different methods (Hotez and Bottazzi, 2021).

Therefore, I hypothesed that the presentation of the antigenic S1 subunit of SARS-CoV2-S protein in large amounts and in its natural structure in the eucaryotic cell membrane and its use as a whole cell or cell membrane will be a new vaccination protocol for the SARS-CoV-2. Presenting the S1 of SARS-CoV2 in large amount and in its natural structure in cell membrane will provide a much stronger and more specific immune response.

Most importantly, in this way it will be possible to prepare an almost unlimited amount of SARS-CoV2-S antigen in its native form using the cell lines in a short time.

In addition, since the S protein will be attached to the cell membrane, it will not be able to circulate freely, so it will not pass the blood-brain barrier and it may not show the cell signal stimulation effects mentioned above.

Whole death tumor cell (autologous or allogeneic) vaccinations in cancer patients have been tested for a long time and its clinical applications have been approved and used. Neller et al. examined the clinical outcomes of 173 published peer-reviewed immunotherapy trials that used melanoma, renal cell and hepatocellular carcinomas, lung, prostate, breast, colorectal, cervical, pancreatic or ovarian cancer. They showed that both allogeneic and authogenic whole tumor cells did not cause any significant side effects (Neller et al. 2008)

Therefore, after the SARS-CoV2-S Δ21 is expressed in allogeneic cancer cells, it will be possible to use it in vaccination, both as a whole cell (death) or cell membrane extraction.

The use of immortal authogenic and/or allogeneic primary tissue cells or stem cells, for expression of SARS-CoV2-S Δ21, is also among the objectives of this invention.

One of the advantage of using live cell expressing SARS-CoV2-S is that the emerging of heat-shock proteins, uric acid, HMGB1 (high-mobility-group box 1), ATP and double-stranded genomic DNA from dying cells have adjuvant effect as shown in previous studies (Radford et al. 2014).

Recently, the use of exosomes in cancer vaccine and treatment has gained importance. Exosomes are nano-sized membrane vesicles derived from the late endosomal compartment, capable of transferring proteins, lipids and RNA between cells. The majority of studies investigating exosomes as a cancer vaccine or therapy have been focusing on whole antigen-loaded exosomes which are able to induce immune reaction (Nicolini, et All., 2021). In addition recent findings showed that B cells and dendritic cells (DC) release exosomes expressing major histocompatibility complex (MHC) class I and II, as well as co-stimulatory molecules (CD80/86) and can induce peptide-specific T cell proliferation in an MHC dependent manner (Liu, et al. 2019).

Therefore, the S protein expression in its natural form and in large amounts in the cell membrane and using it for vaccination purposes is similar to the exosome vaccine protocols. In addition, it is known that opposite to the exosome, all tissues contain MHCI expression in their cell membrane.

### SUMMARY OF THE INVENTION

Exosomes are nano-sized membrane vesicles capable of transferring proteins, lipids and RNA between cells. The whole antigen-loaded exosomes are able to induce immune reaction.

This invention was developed in line with the logic of immune response as result of the transport of protein (antigen) loaded on exosomes. In this invention, instead of antigen transported by exosomes, it based on the transport of protein (antigen) by loading it into cell membrane, the provision of antigen presentation and the induction of an immune response.

The C-terminal moiety of SARS-CoV2-S cytoplasmic tail is essential for retention of S protein in ERGIC compartment. Therefore, in this invention, it was hypothesized that amino acid deletion in the C region of the SARS-CoV2-S protein would be enable the SARS-CoV2-S protein to take place in the cell membrane without remaining in ERGIC. Importantly, they will be presented in the cell membrane in their most natural form.

Therefore, the invention is based on the S2 subsegment protein of SARS-CoV2-S with the last 21 amino acid removed transporting the antigenic protein (S1 subsegment) to the cell membrane and there which it presents it densly (FIG. 1b, c).

According to a first aspect of the present invention, SARS-CoV2 Spike Δ21 overexpression places SARS-CoV2-S protein in the cell membrane (FIG. 1f-j).

Therefore, the present invention provides a whole SARS-CoV2-S protein with the last 21 amino acid removed (Spike Δ21) fragment overexpressed in a eukaryotic cell and presented in/with cell membrane as a novel coronavirus SARS-CoV2 vaccine.

Another embodiment of the present invention is that the expression of the SARS-CoV2-S Δ21 gene was performed using vector systems designed to tetracycline-inducible Retroviral mammalian expression system that is controlled by the addition of doxycycline (Dox) to the culture medium.

The present invention demonstrated that depending on the amount of SARS-CoV2-S protein expressed, cell-cell fusion was observed in Hep3B cell 24 hours after dox adding, while in 48 hours advanced unification of the cells was observed to distrupt the cell structure (FIG. 2a). Although, not as dramatic as in Hep3B cells, SARS-CoV2-S Δ21 expression also creates fusion in Renca and MAD109 cells (FIGS. 1i and j)

The present invention also demonstrated that SARS-CoV2 Spike Δ21 expressing cell provides a large amount of spike protein expression in the membrane of cell determined by ELISA assay in addition to the dotblot and IHC assays (FIG. 3a-e).

Therefore, the present invention provides an ELISA assay to measure of the levels anti-SARS-CoV2-S antibodies (IgM, IgG and IgA) of the serum of the person infected or vaccinated with SARS-CoV2.

The present invention also presented for the first time that as a result of SARS-CoV2-S Δ21 overexpression in the cell, S1 protein is transported to the cell membrane and replaces the major membrane proteins SPTA1 and CDH2. It was determined that the cell membrane proteins including SPTA1 or CDH2 decreased in the cell membrane at a rate similar to the amount of increase of S protein in the cell membrane. These results are demonstrated by dotblot and ELISA (FIG. 4a-h)

The present invention also presented that using allogeneic cancer cells expressing SARS-CoV2-S Δ21 in their membrane as vaccine induce high amount of SARS-CoV2-S protein specific antibody (FIG. 5a, b).

The present invention also demonstrated that the use of viable, death (UV, Freeze-thaw) cell or only cell membranes of allogeneic cells expressing SARS-CoV2-S protein induce high amount of SARS-CoV2-S protein specific antibody (data not shown).

The present invention also demonstrated that their use as a result of obtaining the cell membrane by different methods such as optical, mechanical, electric and chemical lyses techniques also induce high amount of SARS-CoV2-S protein specific antibody (data not shown).

As a result, the mean endpoint titers of S protein specific IgG in mice immunized with a single vaccination of the Renca WhC expressing SARS-CoV2-S (WhC-S) with CFA was significantly higher than those observed in mice that received PBS with CFA (FIG. 5a). Additionally, S protein specific IgG in mice immunized with the CM expressing SARS-CoV2-S(CM-S), although not significant, a slight increase was observed compared to the goup of mice immunized with the WhC-S (FIG. 5c). In addition, adding CFA to immunization, although not significant, slightly increased S protein specific IgG in both WhC-S and CM-S immunizations (FIG. 5d).

It was determined that vaccination with CM-S produced a statistically much stronger immune response than vaccination with BNT162b2. It was also determined that the difference between these two groups of vaccination systems was higher in single dose vaccination (FIG. 5e). In addition, in comparison with the pool of human convalescent patient serum (hcPS), vaccination with CM-S produced a statistically significant stronger immune response even a single dose of CM-S vaccination (FIG. 5e).

In the both neutralization tests with cell membrane presenting SARS-CoV2-S protein (CM-S) and NM coated with RBD unit of SARS-CoV2-S protein, a statistically significant difference was observed in the double vaccination with CM-S compared to the BNT162b2 vaccine and hcPS. All the neutralization tests were completed with sera obtained after the booster dose (FIGS. 6a and b).

Similar to the neutralization tests described above, in Live SARSCoV-2 neutralization assays, a statistically significant difference was observed in the double vaccination with CM-S compared to the BNT162b2 vaccine and hcPS. The mean NT50 (50% neutralization titers) value in the CM-S was 3,8- and 4,2-fold higher than those observed in the BNT162b2 vaccine and the hcPS, respectively (FIG. 6c).

### Statements

In one embodiment of the present invention, the number of amino acid removed from the the S2 subsegment protein of SARS-CoV2-S can be less than 21 amino acid such as, 20, 19, 18, 17, 16, 15, 14, 13, or less amino acid

In another embodiment of the present invention is that the number of amino acid removed from the the S2 subsegment protein of SARS-CoV2-S can be more than 21 amino acid such as, 22, 23, 24, 25, 26, 27, or more amino acid.

Another embodiment of the present invention is that the S2 subunit of the SARS-CoV2-S, which transport the protein which it is fused to the cell membrane, may not need all of its segments including the FP, HR1, HR2, TM, and CT domain to perform transport function to cell membrane.

Another embodiment of the present invention is that the expression of the SARS-CoV2-S Δ21 gene will preferably be performed using vector systems designed to tetracycline-inducible mammalian expression system that is controlled by the addition of Dox to the culture medium. The other inducible vector system can also be applied for this purpose.

Another embodiment of the present invention is that inducible vector system can be recombinant virus related, such as Retrovirus, Lentivirus or Adenovirus associated vector, bacteriophages or any other viral systems.

Another embodiment of the present invention is that a recombinant virus related, such as Retrovirus, Lentivirus or Adenovirus associated vector systems that provide sustained expression of SARS-CoV2-S protein can also be used if their expression do not damage the cell being used.

Another embodiment of the present invention is that the expression of the SARS-CoV2-S Δ21 gene will preferably be performed using vector systems containing EF1 promoter. It is also possible to use CMV and other promoter-containing vector systems that function in mammalian cells

In one embodiment of the present invention is the use cell membrane (CM) prepared as described in this invention, presenting SARS-CoV2-S Δ21 in its structure obtained from the allogeneic cancer cell lines as SARS-CoV2 vaccine.

In one embodiment of the present invention is the use whole death cell (WhC) prepared as described in this invention, presenting SARS-CoV2-S Δ21 in its structure obtained from the allogeneic cancer cell lines as SARS-CoV2 vaccine.

In one embodiment of the present invention is the use WhC (live) prepared as described in this invention, presenting SARS-CoV2-S Δ21 in its membrane (WhC-S) obtained from the allogeneic primary tissue cell lines as SARS-CoV2 vaccine.

In one embodiment of the present invention is the use CM prepared as described in this invention, presenting SARS-CoV2-S Δ21 in its structure (CM-S) obtained from the allogeneic primary tissue cell lines as SARS-CoV2 vaccine.

In one embodiment of the present invention is the use CM-S prepared as described in this invention, presenting SARS-CoV2-S Δ21 in its structure obtained from the primary immortalized allogeneic cell lines as SARS-CoV2 vaccine.

In one embodiment of the present invention is the use live cell, with MHC antigens which produce the allogeneic immune response in recipient, presenting SARS-CoV2-S Δ21 in its structure obtained from the primary immortalized allogeneic cell lines as SARS-CoV2 vaccine.

In one embodiment of the present invention is the use WhC (live) prepared as described in this invention, presenting SARS-CoV2-S Δ21 in its membrane obtained from the allogeneic stem cell lines including Embryonic, Mesenchimal, Fetal, Adult, Amniotic stem cell lines as SARS-CoV2 vaccine.

In one embodiment of the present invention is the use WhC (live) with MHC antigen which produce the allogeneic immune response in recipient, prepared as described in this invention, presenting SARS-CoV2-S Δ21 in its membrane obtained from the allogeneic stem cell lines including Embryonic, Mesenchimal, Fetal, Adult, Amniotic stem cell lines as SARS-CoV2 vaccine.

In one embodiment of the present invention is the use CM-S prepared as described in this invention, presenting SARS-CoV2-S Δ21 in its structure obtained from the allogeneic stem cell including Embryonic, Mesenchimal, Fetal, Adult, Amniotic stem cell lines as SARS-CoV2 vaccine.

In one embodiment of the present invention is the use live cell (allogeneic, primary, immortal) of antigen presenting cells (APCs) including macrophages, B cells, and especially dentritic cells prepared as described in this invention, presenting SARS-CoV2-S Δ21 in its structure, as SARS-CoV2 vaccine. Wherein the APC cells will be selected to elicit an immune response in the recipient due to the MHC antigens they carry.

In one embodiment of the present invention is the use CM-S (allogeneic, secondary, cancer, immortal) prepared as described in this invention, presenting SARS-CoV2-S Δ21 in its structure obtained from the APCs as SARS-CoV2 vaccine.

In one embodiment of the present invention is the use WhC (live) prepared as described in this invention, presenting SARS-CoV2-S Δ21 in its membrane obtained from the autologous stem cell, primary tissue cells (immortalized) lines as SARS-CoV2 vaccine.

In one embodiment of the present invention is the use CM-S prepared as described in this invention, presenting SARS-CoV2-S Δ21 in its structure obtained from the autologous stem cell, primary tissue cell lines as SARS-CoV2 vaccine.

In one embodiment, the present invention provides that expression of SARS-CoV2-S protein on the CM results in the replacement of other CM proteins and reducing them in the CM, which eventually prevent the formation of undesirable background antibodies.

In one embodiment of the present invention is that the use of CM-S expressing SARS-CoV2-S protein as vaccine will prevent the formation of undesirable background antibodies.

The use of present invention, in autogenic or allogeneic cells with SARS-CoV2-S in their membrane for the immunization, will prevent the formation of undesirable background antibodies and will ensure that target SARS-CoV2-S specific pure antibodies are obtained.

In another embodiment, the present invention provides a method for producing antibody against SARS-CoV-2-S proteins of SARS-CoV-2.

In another embodiment the present invention provides a method of immunodiagnostic test kits that identify both antibodies and/or antigens that are disease markers. For example, searching Covid-19 S-specific antibodies by ELISA, in which wells were coated with cell or cell membranes presenting SARS-CoV2-S protein obtained by the method of present invention.

In the methods of this invention in which protein (antigen) and then specific antibody are obtained, a variety of assays can be employed. For example, various immunoassays can be used to detect antibodies or proteins (antigens) of this invention. Such immunoassays typically involve the measurement of antigen/antibody complex formation between a protein or peptide (i.e., an antigen) and its specific antibody.

According to another aspect of the present invention provides the opportunity to use cells expressing SARS-CoV2-S protein in its membrane grown in cell culture plate for ELISA test. In this test, the permeabilization would not be necessary, as the SARS-CoV2-S protein is intensly and naturally presented in the cell membrane (FIG. 3a-d).

According to another aspect of the present invention provides the opportunity to use cell membrane containing SARS-CoV2-S Δ21 in development of the ELISA system or other antibody screening tests. In this test the cell membrane which contains a large amount of SARS-CoV2-S protein in its natural structure will be used coating the ELISA plate.

According to another aspect of the present invention provides the opportunity to use cells membranes containing SARS-CoV2-S protein to use in antibody production for S protein. Wherein the cell presenting the SARS-CoV2-S protein in its membrane can be allogeneic.

In another aspect of the present invention provides the opportunity to use whole allogeneic cancer, primary, stem cells prepared as described in this invention, presenting a large amount of SARS-CoV2-S protein in its natural structure in its membrane will be used for the antibody production for S protein.

In one embodiment of the present invention is the use of live cell of allogeneic cells (cancer, primary, stem cells) prepared as described in this invention, containing SARS-CoV2-S Δ21 in its structure to be used in antibody production for S protein. Wherein the cells will be selected to elicit an immune response in the recipient due to the MHC antigens they carry.

In one embodiment of the present invention is the use of live cell of APCs including macrophages, B cells, and especially dentritic cells prepared as described in this invention, containing SARS-CoV2-S Δ21 in its membrane to be used in antibody production for S protein. Wherein the APC cells will be selected to elicit an immune response in the recipient due to the MHC antigens they carry.

The scope of the present invention also include all nucleotide changes that may occur in either S1 or S2 as a result of the codon optomization algorithm. The nucleic acid sequence derivatives of the invention have at least 30%, 40%, 50%, 60%, 70%, 80%, 85%, 90%, 91%, 92%, 93%, 94%, 95%, 96%, 97%, 98%, 99% sequence identity to the native nucleic acid sequence.

The scope of the present invention also include the followings;

It is also possible to present different epitops of SARS-CoV2-S1 including RBD or parts of RBD or capsid proten by fusing coding sequences of different epitops of S1 to S2 to present at the cell membrane.

The scope of the present invention is not intended to be limited to the SARS-CoV2 related vaccination procedure, antibody production, development of the ELISA sytem or other antibody screening test. The invention provides a vaccination procedure, antibody production, development of the ELISA sytem or other antibody screening tests procedures as it enables the antigenic epitops of all viruses to be presented in the cell membrane via the SARS-CoV2-S2 protein.

Therefore, invention covers all viruses including HIV as a result of fusing any viral protein with antigenic properties with the SARS-CoV2-S2 protein, ensuring their expression in the cell and transpoting them to the cell membrane with the help of SARS-CoV2-S2 and present there intensively.

In another embodiment, the present invention provides vaccination procedure, antibody production, development of the ELISA sytem or other antibody screening tests procedures as a result of fusing any bacterial protein with antigenic properties with the S2 protein, ensuring their expression in the cell and transporting them to the cell membrane with the help of S2 and present there intensively.

Therefore, invention covers vaccination protocol for all bacteria as a result of fusing any bacterial protein with antigenic properties with the SARS-CoV2-S2 protein, ensuring their expression in the cell and transpoting them to the cell membrane with the help of SARS-CoV2-S2 and presents there intensively.

The present invention further provides, in another aspect, a method for treating or preventing viral infection, providing a vaccination protocol as a result of fusing multiple viral proteins (more than one in the same cell membrane) showing different antigenic structures with the SARS-CoV2-S2 protein, ensuring their expression in the cell and transporting them to the cell membrane with the help of SARS-CoV2-S2 protein and presents there intensively to the immune system.

The present invention further provides, in another aspect, a method for treating or preventing bacterial infection, providing a vaccination protocol as a result of fusing multiple bacterial proteins showing different antigenic structure with the S2 protein, ensuring their expression in the cell and transporting them to the cell membrane with the help of S2 and presents itself there intensively to the immune system.

Where the bacterial or viral associated proteins do not need to be whole, it is possible for smaller epitops showing antigenic proporties to be presented by binding to the S2 subunit of SARS-CoV2-S.

Therefore, the present invention further provides, in another aspect, a method, in which it is possible to present more than one antigenic structure of the virus or bacteria by using the S2 subunit of SARS-CoV2-S. This will naturally lead to a much stronger vaccination protocol.

The present invention further provides, in another aspect, a method for treating or preventing cancer, providing a vaccination protocol as a result of fusing tumor associated proteins with the S2 protein, ensuring their expression in the cell and transporting them to the cell membrane with the help of S2 and therefore present itself there intensively to the immune system. In here, tumor antigen expressed cell could be living autologus primary stem cell, allogeneic primary stem cell and cancer cell lines with different MHC antigens from the donor. Furthermore, all cell types can be used in the ectopic expression of tumor associated proteins and its transporting by the help of S2 protein and incorporation into the CM and then using the CM as a natural carrier.

The present invention further provides, in another aspect, a method for treating or preventing cancer, providing a much stronger vaccination protocol as a result of fusing multiple tumor associated proteins (or epitops) showing different antigenic structure with the SARS-CoV2-S2 protein, ensuring their expression in the cell and transporting them to the cell membrane with the help of S2 and therefore presents itself there intensively to the immune system.

Although, the present invention described S2 subunit of SARS-CoV2-S protein as a antigenic protein carrier, different DNA sequences of plasma membrane proteins such as Vesicular stomatitis virus G (VSV-G) which is a well-studied plasma membrane protein can also perform the same function (Miyanohara 2012). Therefore, the scope of the present invention is not limited to use of the S2 subunit of SARS-CoV2-S protein as a antigenic epitop carrier, the other protein molecule or molecules that carry proteins to the cell membrane in cell also can be used for the same purpose.

### DETAILED DESCRIPTION OF THE INVENTION

Although the present invention is described in detail below, it is to be understood that this invention is not limited to the particular methodologies, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments, however, it should be understood that they may be combined in any manner and in any number to create additional embodiments. The variously described examples and preferred embodiments should not be interpreted to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

The practice of the present invention will employ, unless otherwise indicated, conventional methods of chemistry, biochemistry, and recombinant DNA techniques which are explained in the literature in the field (e.g., Molecular Cloning: A Laboratory Manual, 3^{nd} Edition, Sambrook and Russel eds., Cold Spring Harbor Laboratory Press, Cold Spring Harbor 2001).

Throughout this specification and the claims which follow, unless the context requires otherwise, the word “comprise”, and variations such as “comprises” and “comprising”, will be understood to imply the inclusion of a stated member. The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”), provided herein is intended merely to better illustrate the invention and does not pose a limitation on the scope of the invention otherwise claimed. No language in the specification should be construed as indicating any non-claimed element essential to the practice of the invention.

Several documents are cited throughout the text of this specification. Each of the documents cited herein, whether supra or infra, are hereby incorporated by reference in their entirety. Nothing herein is to be construed as an admission that the invention is not entitled to antedate such disclosure by virtue of prior invention.

The present invention provides a method that is based on the deletion of 21 amino acids from the C terminal region of the S2 subunit of SARS-CoV2 Spike protein (SARS-CoV2-S Δ21) and transporting the S1 subunit, which is fused to S2 in nature to the cell membrane (CM).

The present invention provides a nucleic acid molecule encoding the chimeric S1 subunits fused to S2 Δ21 subunit of SARS-CoV2-S protein. Chimeric S1 can be prepared to include all of the active mutations identified so far and that may occur later.

The chimeric SARS-CoV2-S proteins of this invention can be produced as recombinant proteins in a eukaryotic cell system and presented in the CM.

The invention also provides immunogenic compositions comprising the cells with chimeric SARS-CoV2-S Δ21, or CM with SARS-CoV2-S Δ21.

The present invention further provides a method of producing an immune response to SARS-CoV2-S Δ21 in a subject, comprising administering to the subject an effective amount of cell or CM with a chimeric SARS-CoV2-S Δ21 protein, including any combination thereof, thereby producing an immune response to a SARS-CoV2 in the subject.

The present invention also provides a method of treating and/or preventing a coronavirus infection in a subject, comprising administering to the subject an effective amount of a cell with SARS-CoV2-S Δ21, and/or CM with SARS-CoV2-S Δ21 composition of this invention.

Additionally provided herein is a method of preventing a disease or disorder caused by a SARS-CoV2 infection in a subject, comprising administering to the subject an effective amount of cell or CM with the SARS-CoV2-S Δ21 protein including any combination thereof, thereby preventing a disease or disorder caused by a coronavirus infection in the subject.

Furthermore, the present invention provides a method of protecting a subject from the effects of SARS-CoV2 infection, comprising administering to the subject an effective amount of cell or CM with the SARS-CoV2-S protein, including any combination thereof, thereby protecting the subject from the effects of coronavirus infection.

Thus, the present invention provides a method of inducing or eliciting an immune response in a subject, comprising administering to the subject an effective amount of a cell with SARS-CoV2 S, and/or CM with SARS-CoV2-S composition of this invention.

Therefore, the present invention provides a vaccination procedure which can be used to immunize a subject against infection by SARS-CoV-2, as well as vaccinate a subject for a newly formed (mutant) SARS-CoV-2.

A subject of this invention is any mammal that is capable of producing an immune response against a coronavirus. A subject of this invention can be a mammal and in particular embodiments is a human, which can be an infant, a child, an adult or an elderly adult. The terms “subject” and “individual” are used interchangeably and relate to mammals. For example, mammals in the context of the present invention are humans, non-human primates, domesticated animals such as dogs, cats, sheep, cattle, goats, pigs, horses etc., laboratory animals such as mice, rats, rabbits, and any others. The term “animal” as used herein also includes humans. The term “subject” may also include a patient, i.e., an animal, preferably a human having a disease. A “subject at risk of infection by a coronavirus” or a “subject at risk of coronavirus infection” is any subject who may be or has been exposed to a coronavirus.

The cell or CM thereof of this invention can be administered, delivered and/or introduced into the subject according to any method now known or later identified for administration, introduction and/or delivery of cell or CM, as would be well known to one of ordinary skill in the art.

The term “cell membrane” (also known as the plasma membrane or cytoplasmic membrane, and historically referred to as the plasmalemma) is a biological membrane that separates the interior of all cells from the outside environment which protects the cell from its environment. The cell membrane consists of a lipid bilayer that sit between phospholipids to maintain their fluidity at various temperatures. A variety of protein receptors and identification proteins, such as antigens, are present on the surface of the membrane. Most membrane proteins must be inserted in some way into the membrane. For this to occur, an N-terminus “signal sequence” of amino acids directs proteins to the endoplasmic reticulum, which inserts the proteins into a lipid bilayer. Once inserted, the proteins are then transported to their final destination in vesicles, where the vesicle fuses with the target membrane.

The term “exosome” is nano-sized membrane vesicles derived from the late endosomal compartment. Cells release exosome into the extracellular environment which represent an important mode of intercellular communication by serving as vehicles, capable of transferring proteins, lipids and RNA. Therefore antigen-loaded exosomes are used as a cancer vaccine method in new studies.

Thus, similar to the use of exosome in vaccination, the present invention provides a method that use CM as carrier molecules which carry antigens, as in the example of vaccination protocol created by presenting the SARS-CoV2-S protein on the CM in this invention.

The exact amount of the cell with SARS-CoV2-S or CM with SARS-CoV2-S required will vary from subject to subject, depending on the species, age, weight and general condition of the subject, the particular primary autogenic or allogenic cell used, its mode of administration and the like. Thus, it is not possible to specify an exact amount for every cell. However, an appropriate amount can be determined by one of ordinary skill in the art using only routine experimentation given the teachings herein. As used herein, an “effective amount” refers to an amount of a compound or composition that is sufficient to produce a desired effect, which can be a therapeutic, prophylactic and/or beneficial effect. One nonlimiting example of an effective amount of a cell or CM per cell of this invention is from about 10³ to about 10¹⁰ cell, preferably from about 10³ to about 10⁹, and in particular from about 10⁴ to about 10⁶ cell or CM, per cell, which can be administered to a subject, depending upon the age, species and/or condition of the subject being treated. As would be well known to one of ordinary skill in the art, the optimal dosage would need to be determined for any given antigen or vaccine, e.g., according to the method of production and resulting immune response.

Ideally, a subject will receive a single injection. If additional injections are necessary, they can be repeated at weekly/monthly intervals for an indefinite period and/or until the efficacy of the treatment has been established. As set forth herein, the efficacy of treatment can be determined by evaluating the symptoms and clinical parameters and/or by detecting a desired immunological response.

Embodiments of the present invention include antibodies produced a subject of this invention that has produced an immune response against the SARS-CoV2-S protein of this invention, wherein said antibodies are specific to epitopes present on the SARS-CoV2-S protein. Such antibodies can be specific for an epitope in any parts of SARS-CoV2-S protein of this invention as described herein.

The cell or CM may be delivered subdermally, intradermal or intramuscular. The most suitable route in any given case will depend, as is well known in the art, on such factors as the species, age, gender and overall condition of the subject, the nature and severity of the condition being treated and/or on the nature of the particular composition (i.e., cell, CM, dosage-cell, CM/per cell) that is being administered.

The scope of the present invention is not intended to be limited to the SARS-CoV2-S related vaccination procedure, as a result of the formation of the chimeric structure formed by the SARS-CoV2 S2 subunit, which act as a carrier to CM, and any viral protein with antigenic properties, a vaccination protocol can be prepared to cover all viruses. Therefore, invention covers all viruses including HIV.

The scope of the present invention, therefore, cover vaccination procedure for the all infectious disease, as a result of the formation of the chimeric structure formed by the SARS-CoV2 S2 subunit, which act as a carrier to CM, and a protein with antigenic properties of the any infectious agents (disease-associated antigen).

The term “disease” or “disorder” refers to an abnormal condition that affects the body of an individual. A disease is often construed as a medical condition associated with specific symptoms and signs. A disease may be caused by factors originally from an external source, such as infectious disease, or it may be caused by internal dysfunctions, such as autoimmune diseases. As used herein the term “disease” or “disorder” includes, in particular, a condition which would benefit from the expression of a peptide or protein (as described above), e.g, as demonstrated by a reduction in and/or an amelioration of symptoms.

The term “infectious disease” refers to any disease which can be transmitted from individual to individual or from organism to organism, and is caused by a microbial agent (e.g. common cold). Examples of infectious diseases include viral infectious diseases, such as AIDS (HIV), Coronavirus, hepatitis A, B or C, herpes, herpes zoster (chicken-pox), German measles (rubella virus), yellow fever, dengue etc. flaviviruses, influenza viruses, hemorrhagic infectious diseases (Marburg or Ebola viruses), and severe acute respiratory syndrome (SARS), bacterial infectious diseases, such as Legionnaire's disease (Legionella), sexually transmitted diseases (e.g. chlamydia or gonorrhea), gastric ulcer (Helicobacter), cholera (Vibrio), tuberculosis, diphtheria, infections by E. coli, Staphylococci, Salmonella or Streptococci (tetanus); infections by protozoan pathogens such as malaria, sleeping sickness, leishmaniasis; toxoplasmosis, i.e. infections by Plasmodium, Trypanosoma, Leishmania and Toxoplasma; or fungal infections, which are caused e.g. by Cryptococcus neoformans, Histoplasma capsulatum, Coccidioides immitis, Blastomyces dermatitidis or Candida albicans.

The term “disease-associated antigen” is used in it broadest sense to refer to any antigen associated with a disease. A disease-associated antigen is a molecule which contains epitopes that will stimulate a host's immune system to make a cellular antigen-specific immune response and/or a humoral antibody response against the disease. The disease-associated antigen may therefore be used for therapeutic purposes. Disease-associated antigens are preferably associated with infection by microbes, typically microbial antigens, or associated with cancer, typically tumors. The antigen may be a disease-associated antigen, such as a tumor-associated antigen, a viral antigen, or a bacterial antigen.

According to the invention, the term “disease” also refers to cancer diseases. The terms “cancer disease” or “cancer” (medical term: malignant neoplasm) refer to a class of diseases in which a group of cells display uncontrolled growth (division beyond the normal limits), invasion (intrusion on and destruction of adjacent tissues), and sometimes metastasis (spread to other locations in the body via lymph or blood). These three malignant properties of cancers differentiate them from benign tumors, which are self-limited, and do not invade or metastasize. Most cancers form a tumor, i.e. a swelling or lesion formed by an abnormal growth of cells (called neoplastic cells or tumor cells), but some, like leukemia, do not. Examples of cancers include, but are not limited to, carcinoma, lymphoma, blastoma, sarcoma, glioma and leukemia. More particularly, examples of such cancers include bone cancer, blood cancer, lung cancer, liver cancer, pancreatic cancer, skin cancer, cancer of the head or neck, cutaneous or intraocular malignant melanoma, uterine cancer, ovarian cancer, rectal cancer, cancer of the anal region, stomach cancer, colon cancer, breast cancer, prostate cancer, uterine cancer, carcinoma of the sexual and reproductive organs, Hodgkin's disease, cancer of the esophagus, cancer of the small intestine, cancer of the endocrine system, cancer of the thyroid gland, cancer of the parathyroid gland, cancer of the adrenal gland, sarcoma of soft tissue, cancer of the bladder, cancer of the kidney, renal cell carcinoma, carcinoma of the renal pelvis, neoplasms of the central nervous system (CNS), neuroectodermal cancer, spinal axis tumors, glioma, meningioma, and pituitary adenoma. The term “cancer” according to the invention also comprises cancer metastases.

The term “disease involving an antigen” refers to any disease which implicates an antigen, e.g. a disease which is characterized by the presence and/or expression of an antigen. The disease involving an antigen can be an infectious disease, an autoimmune disease, or a cancer disease or simply cancer. As mentioned above, the antigen may be a disease-associated antigen, such as a tumor-associated antigen, a viral antigen, or a bacterial antigen.

Therefore, the scope of the present invention also cover vaccination procedure for the all tumors, as a result of the formation of the chimeric structure formed by the SARS-CoV2 S2 subunit, which act as a carrier to CM, and any protein with antigenic properties of the tumor (tumor-associated antigen).

The term “viral antigen” refers to any viral component having antigenic properties, i.e. being able to provoke an immune response in an individual. The viral antigen may be a viral ribonucleoprotein or an envelope protein.

The term “bacterial antigen” refers to any bacterial component having antigenic properties, i.e. being able to provoke an immune response in an individual. The bacterial antigen may be derived from the cell wall or cytoplasm membrane of the bacterium.

The present invention further provides, in another aspect, a method for treating or preventing viral, bacterial infection, providing a vaccination protocol as a result of fusing multiple viral or bacterial proteins showing different antigenic structure with the S2 protein, ensuring their expression in the cell and transporting them to the CM with the help of S2 and present there intensively to the immune system.

Where the bacterial or viral associated proteins need not be whole, it is possible for smaller epitops showing antigenic proporties to be presented by binding to the S2 subunit of SARS-CoV2-S.

Therefore, the present invention further provides, in another aspect, a method, in which it is possible to present more than one antigenic structure of the virus or bacteria by using the S2 subunit of SARS-CoV2 S. This will naturally lead to a much stronger vaccination protocol.

The present invention further provides, in another aspect, a method for treating or preventing cancer, providing a much stronger vaccination protocol as a result of fusing multiple tumor associated proteins (or epitops) showing different antigenic structure with the S2 subunit of SARS-CoV2-S protein, ensuring their expression in the cell and transporting them to the CM with the help of S2 and present there intensively to the immune system. This process can be applied by fusing different antigenic proteins or peptides to the SARS-CoV2-S2 protein in a single vector system in frame shift. This process also can be achived in the same cell using more than one vector systems with different selection markers to express each antigenic protein fused to SARS-CoV2-S2 protein.

The term “immune response”, as used herein, relates to a reaction of the immune system such as to immunogenic organisms, such as bacteria or viruses, cells or substances. The term “immune response” includes the innate immune response and the adaptive immune response. Preferably, the immune response is related to an activation of immune cells, an induction of cytokine biosynthesis and/or antibody production. It is preferred that the immune response comprises the steps of activation of antigen presenting cells, such as dendritic cells and/or macrophages, presentation of an antigen or fragment thereof by said antigen presenting cells and activation of cytotoxic T cells due to this presentation.

In another embodiment, the present invention provides a method for producing an antibody. The antibody is useful as a therapeutic agent, diagnostic agent or reagent for various diseases. As a general method for producing an antibody, a method in which an antigen is administered to a mammal such as a mouse and the antibody is obtained from the serum of the animal is used. In the production of antibodies, the target antibody may not be obtained efficiently. The two main difficulties are that the amount of antigen is not enough to immunize mammals and it is not sufficiently purified. Therefore, it is desirable to prepare a large amount of sufficiently purified antigen for immunization. However, in reality, many antigens are difficult to purify and it is difficult to prepare a sufficient amount. In other words, the antigen preparation process has often been an obstacle in antibody production. As a method for expressing a large amount of protein, a method using a baculovirus has attracted attention. By using these methods, a desired membrane protein can be expressed in large quantities on a baculovirus membrane.

However, a baculovirus-derived membrane protein is expressed on the baculovirus membrane thus obtained in addition to the foreign membrane protein (a background antigen). Therefore, when an antibody is produced using a budding baculovirus as an immunogen, an antibody against a baculovirus-derived membrane protein is also produced. As a result, it has been difficult to efficiently produce an antibody against the target membrane protein by a known immunization method. Therefore, in order to use the target membrane protein expressed on the baculovirus membrane as an antigen, it is necessary to sufficiently purify the membrane protein. However, it is generally difficult to purify foreign membrane proteins from budding baculoviruses. Therefore, it can be said that it is impossible in practice to obtain a highly purified membrane protein sufficient for immunization. For antigens that are difficult to purify, it has been difficult to obtain the desired antibody by conventional methods.

In one embodiment of the present invention is that the use of CM expressing SARS-CoV2-S protein as vaccine will prevent the formation of undesirable background antibodies.

Importantly, the present invention provides that expression of SARS-CoV2-S protein on the CM results in the replacement of other CM proteins and reducing them in the CM, which eventually prevent presentation of other CM proteins and the formation of undesirable background antibodies.

In additon, the use of present invention, in autogenic or allogeneic cells with antigen in their membrane to the immunized animal, will prevent the formation of undesirable background antibodies and will ensure that target antigen specific pure antibody is obtained. As stated above, the most important benefit of the present invention is obtaining a large amount of protein in its natural structure. Therefore the present invention will solve the problems mentioned above (antibody production using baculovirus) by obtaining and using a high amount of pure and natural protein presented in CM for immunization.

Therefore, the present invention provides a method for producing antibodies against proteins of all disease-associated egents, including, viruses, bacteria, tumor and other diseases.

In another embodiment the present invention provides a method of immunodiagnostic test kits that identify both antibodies and/or antigens that are disease markers. For example, searching SARS-CoV2-S specific antibodies by enzyme-linked immunosorbent assays (ELISA) assay, in which wells were coated with cell or CM presenting SARS-CoV2-S proteins obtained by the method of the present invention is an important and frequently used immunodiagnostic method.

In the methods of this invention in which protein (antigen) and then specific antibody are obtained, a variety of assays can be employed. For example, various immunoassays can be used to detect antibodies or proteins (antigens) of this invention. Such immunoassays typically involve the measurement of antigen/antibody complex formation between a protein or peptide (i.e., an antigen) and its specific antibody.

The immunoassays of the invention can be either competitive or noncompetitive and both types of assays are well-known and well-developed in the art. In competitive binding assays, antigen or antibody competes with a detectably labeled antigen or antibody for specific binding to a capture site bound to a solid surface. The concentration of labeled antigen or antibody bound to the capture agent is inversely proportional to the amount of free antigen or antibody present in the sample.

In some embodiments, antibodies and/or proteins can be conjugated or otherwise linked or connected (e.g., covalently or noncovalently) to a solid support (e.g., bead, plate, slide, dish, membrane or well) in accordance with known techniques. Antibodies can also be conjugated or otherwise linked or connected to detectable groups such as radiolabels (e.g., ³⁵S, ¹²⁵I, ³²P, ¹³H, ¹⁴C, ¹³¹I), enzyme labels (e.g., horseradish peroxidase, alkaline phosphatase), gold beads, chemiluminescence labels, ligands (e.g., biotin) and/or fluorescence labels (e.g., fluorescein) in accordance with known techniques.

A variety of organic and inorganic polymers, both natural and synthetic can be used as the material for the solid surface. Nonlimiting examples of polymers include polyethylene, polypropylene, poly(4-methylbutene), polystyrene, polymethacrylate, poly(ethylene terephthalate), rayon, nylon, poly(vinyl butyrate), polyvinylidene difluoride, silicones, polyformaldehyde, cellulose, cellulose acetate, nitrocellulose, and the like. Other materials that can be used include, but are not limited to, paper, glass, ceramic, metal, metalloids, semiconductive materials, cements and the like. In addition, substances that form gels, such as proteins (e.g., gelatins), lipopolysaccharides, silicates, agarose and polyacrylamides can be used. Polymers that form several aqueous phases, such as dextrans, polyalkylene glycols or surfactants, such as phospholipids, long chain (12-24 carbon atoms) alkyl ammonium salts and the like are also suitable. Where the solid surface is porous, various pore sizes can be employed depending upon the nature of the system.

A variety of immunoassay systems can be used, including but not limited to, radio-immunoassays (RIA), ELISA assays, enzyme immunoassays (EIA), “sandwich” assays, gel diffusion precipitation reactions, immunodiffusion assays, agglutination assays, immunofluorescence assays, fluorescence activated cell sorting (FACS) assays, immunohistochemical assays, protein A immunoassays, protein G immunoassays, protein L immunoassays, biotin/avidin assays, biotin/streptavidin assays, immunoelectrophoresis assays, precipitation/flocculation reactions, immunoblots (Western blot; dot/slot blot); immunodiffusion assays; liposome immunoassay, chemiluminescence assays, library screens, expression arrays, immunoprecipitation, competitive binding assays and immunohistochemical staining. These and other assays are described, among other places, in Hampton et al. (Serological Methods, a Laboratory Manual, APS Press, St Paul, Minn. (1990)) and Brown T A (Genomes. 2nd edition. Oxford, Wiley-Liss:2002), Auld et al. (The assay Guidance Manual; https://www.ncbi.nlm.nih.gov/books/).

As mentioned above, the present invention provides a method for producing antibodies against proteins of all disease-associated egents, including, viruses, bacteria, tumor and other diseases.

Therefore, in another embodiment, the present invention provides a method passive antibody tereatment against to viruses, bacteria and other agents.

In passive antibody treatment, specific antibodies required to kill a specific agent are directly delivered into the body.

The term “antibody” or “antibodies” as used herein refers to all types of immunoglobulins, including IgG, IgM, IgA, IgD, and IgE. The antibody can be monoclonal or polyclonal and can be of any species of origin, including, for example, mouse, rat, rabbit, horse, goat, sheep or human, or can be a chimeric or humanized antibody.

According to the invention, a nucleic acid or nucleic acid molecule refers to a nucleic acid which is preferably deoxyribonucleic acid (DNA) or ribonucleic acid (RNA). According to the invention, nucleic acids comprise genomic DNA, cDNA, mRNA, recombinantly prepared and chemically synthesized molecules. According to the invention, a nucleic acid may be in the form of a single-stranded or double-stranded and linear or covalently closed circular molecule. The term “nucleic acid” according to the invention also comprises a chemical derivatization of a nucleic acid on a nucleotide base, on the sugar or on the phosphate.

In a preferred embodiment, a nucleic acid molecule according to the invention is a vector. The term “vector” is used here in its most general meaning and comprises any intermediate vehicles for a nucleic acid which, for example, enable said nucleic acid to be introduced into prokaryotic and/or eukaryotic host cells and, where appropriate, to be integrated into a genome. Such vectors are preferably replicated and/or expressed in the cell. Vectors comprise plasmids, phagemids or virus genomes. The term “plasmid”, as used herein, generally relates to a construct of extrachromosomal genetic material, usually a circular DNA duplex, which can replicate independently of chromosomal DNA. In a preferred embodiment of the present invention is the use of the recombinant virus techniques. They provide a useful alternative to non-viral methods, which have low transfer efficiencies in certain cell types—for example, primary culture and epithelial cells. The most frequently used vectors are retroviral, lentiviral or adenoviral; however, others include adeno associated virus (AAV), herpes virus, vaccinia virus, and several RNA viruses.

Viral techniques of gene transfer use the method of entry and integration with the host genome used by the wild-type organism.

Although adenoviral vectors are useful in transient assays, retroviral vectors stably integrate into the dividing target cell genome so that the introduced gene is passed on and expressed in all daughter cells.

Another embodiment of the present invention is that the expression of the SARS-CoV2-S Δ21 gene or the gene products with antigenic properties to be prepared for any disease as described in the text will preferably be performed using vector systems containing EF1 promoter. The term “promoter” or “promoter region” refers to a DNA sequence upstream (5′) of the coding sequence of a gene, which controls expression of said coding sequence by providing a recognition and binding site for RNA polymerase. The promoter region may include further recognition or binding sites for further factors involved in regulating transcription of said gene. Examples of promoters preferred according to the invention are promoters for EF1, CMV, pGK and others that function in mammalian cells. A promoter may control transcription of a prokaryotic or eukaryotic gene.

A promoter may be “inducible” and initiate transcription in response to an inducer, or may be “constitutive” if transcription is not controlled by an inducer. An inducible promoter is expressed only to a very small extent or not at all, if an inducer is absent. In the presence of the inducer, the gene is “switched on” or the level of transcription is increased. This is usually mediated by binding of a specific transcription factor. The preferred embodiment of the present invention is the inducible expression of the target gene, here SARS-CoV2 S. The inducible system can be used within plasmid or viral vector systems. Although the tetracyclin inducible vector system is used, this invention also comprise other induction systems.

Another embodiment of the present invention is that a recombinant virus related, such as Retrovirus, Lentivirus or Adenovirus associated vector systems that provide sustained expression of SARS-CoV2-S Δ21 protein can also be used if their expression do not damage the cell being used.

The term “isolated nucleic acid” means according to the invention that the nucleic acid has been (i) amplified in vitro, for example by polymerase chain reaction (PCR), (ii) recombinantly produced by cloning, (iii) purified, for example by cleavage and gel-electrophoretic fractionation, or (iv) synthesized, for example by chemical synthesis. An isolated nucleic acid is a nucleic acid available to manipulation by recombinant DNA techniques. (e.g., as described in Sambrook et al., eds., “Molecular Cloning: A Laboratory Manual,” 3nd ed., Cold Spring Harbor Laboratory, Cold Spring Harbor Laboratory Press, Cold Spring Harbor, N.Y., 2001).

The term “recombinant” in the context of the present invention means “made through genetic engineering”. Preferably, a “recombinant object” such as a recombinant nucleic acid in the context of the present invention is not occurring naturally.

As a nucleic acid, in particular DNA, for expression of more than one peptide or protein, either of a nucleic acid type in which the different peptides or proteins are encoded in different nucleic acid molecules or a nucleic acid type in which the peptides or proteins are encoded in the same nucleic acid molecule can be used.

The term “expression” is used according to the invention in its most general meaning and comprises the production of RNA and/or peptides or proteins, e.g. by transcription and/or translation. With respect to RNA, the term “expression” or “translation” relates in particular to the production of peptides or proteins. It also comprises partial expression of nucleic acids. Moreover, expression can be transient, stable, and/or stable-inducible.

The term “translation” according to the invention relates to the process in the ribosomes of a cell by which a strand of messenger RNA directs the assembly of a sequence of amino acids to make a peptide or protein.

Terms such as “enhancement of expression”, “enhanced expression” or “overexpression” mean in the context of the present invention that the amount of peptide or protein expressed by a given number of vector molecules is higher than the amount of peptide or protein expressed by the same number of vector molecules. The amount of peptide or protein may be given in moles, or by weight, e.g. in grams, or by mass or by polypeptide activity, e.g. if the peptide or protein is an enzyme it may be given as catalytic activity or if the peptide or protein is an antibody or antigen or a receptor it may be given as binding affinity. In one embodiment, terms such as “enhancement of expression”, “enhanced expression” or “increased expression” mean in the context of the present invention that the amount of peptide or protein expressed by a given number of vector molecules and within a given period of time is higher than the amount of peptide or protein expressed by the same number of vector molecules and within the same period of time.

The term “codon optomization” is approach in gene engineering to improve gene expression by changing synonymous codon based on an organism's codon bias. As used herein, “improve” means that the codon-optimized nucleotid sequence used in the non-native cell produces more protein than the non-codon-optimized native nucleotide sequence. As used herein, “nucleotide sequence” describes the expression of an entire protein, or any sequence within the same frame, in a vector, either as an amino acid epitope or as a whole protein.

The level of expression and/or duration of expression of RNA may be determined by measuring the amount, such as the total amount expressed and/or the amount expressed in a given time period, and/or the time of expression of the peptide or protein encoded by the RNA, for example, by using an ELISA procedure, an immunohistochemistry procedure, a quantitative image analysis procedure, a Western Blot, mass spectrometry, a quantitative immunohistochemistry procedure, or an enzymatic assay.

The term “transfection” relates to the introduction of nucleic acids, in particular DNA, into a cell. For purposes of the present invention, the term “transfection” also includes the introduction of a nucleic acid into a cell or the uptake of a nucleic acid by such cell. The term “transduction” relates to Retroviruses that have been used as vehicles for transferring genes into eukaryotic cells, a process known as transduction. Thus, according to the present invention, a cell for transfection of a nucleic acid can be present in vitro or in vivo, e.g. the cell can form part of an organ. For some applications of transfection, it is sufficient if the transfected genetic material is only transiently expressed. Since the nucleic acid introduced in the transfection process is usually not integrated into the nuclear genome, the foreign nucleic acid will be diluted through mitosis or degraded. Cells allowing episomal amplification of nucleic acids greatly reduce the rate of dilution. If it is desired that the transfected nucleic acid actually remains in the genome of the cell and its daughter cells, a stable transfection must occur as in this invention.

According to the present invention, any technique useful for introducing, i.e. transferring or transfecting, nucleic acids into cells in vitro may be used. Preferably, nucleic acid is transfected into cells by standard techniques. Such techniques include Calcium Phosphate-DNA precipitation, electroporation, lipofection and microinjection.

According to the invention it is preferred that introduction of nucleic acid encoding a protein or peptide into cells or uptake of nucleic acid encoding a protein or peptide by cells results in expression of said protein or peptide. The cell may express the encoded peptide or protein intracellularly (e.g. in the cytoplasm and/or in the nucleus), or as in this invention may transport the encoded peptide or protein on the CM and present.

According to the invention, the term “host cell” refers to any cell which can be transformed or transfected with an exogenous nucleic acid. Particular preference is given to mammalian cells such as cells from humans, mice, hamsters, pigs, goats, primates. The cells may be derived from a multiplicity of tissue types and comprise primary cells and cell lines. Specific examples include fibroblast, peripheral blood leukocytes, bone marrow stem cells and embryonic stem cells. A stem cell line is a group of stem cells that is cultured in vitro and can be propagated indefinitely. Stem cell lines are derived from either animal or human tissues and come from one of three sources: embryonic stem cells, adult stem cells, or induced stem cells.

In other embodiments, the host cell is an antigen-presenting cell, in particular a dendritic cell, a monocyte or a macrophage.

According to the invention, the host cells may be derived from immortalized primary cells which are derivatives of primary cells that owing to mutation events end up evading normal cellular senescence and acquiring the ability of continuous cell-division. Immortalized cell lines can be generated from cells isolated from tumors, or mutations can be introduced to make the primary cells immortal. Immortality of cell lines could be achieved by different approaches, including ectopic expression of telomerase or telemorase reverse transcriptase (TERT), by mutating the p53 and pRb genes, or introducing the oncogenes. Viral vectors may be used for all the mentioned approaches, that is introduction of TERT and oncogenes or mutating the p53/pRb.

The term “pharmaceutically active peptide or protein” includes a peptide or protein that can be used in the treatment of a subject where the expression of a peptide or protein would be of benefit, e.g., in ameliorating the symptoms of a disease or disorder. The term “immunologically active compound” relates to any compound altering an immune response, preferably by inducing and/or suppressing maturation of immune cells, inducing and/or suppressing cytokine biosynthesis, and/or altering humoral immunity by stimulating antibody production by B cells. Immunologically active compounds possess potent immunostimulating activity including, but not limited to, antiviral and antitumor activity, and can also down-regulate other aspects of the immune response, for example shifting the immune response away from a TH2 immune response, which is useful for treating a wide range of TH2 mediated diseases. Immunologically active compounds can be useful as vaccine adjuvants.

In some embodiments of the present invention, the compositions can be administered with an adjuvant. As used herein, “adjuvant” describes a substance, which can be any immunomodulating substance capable of being combined with the polypeptide or nucleic acid vaccine to enhance, improve or otherwise modulate an immune response in a subject without deleterious effect on the subject.

Non-limiting examples of adjuvants that can be used in the vaccine of the present invention include the RIBI adjuvant system (Ribi Inc., Hamilton, Mont.), alum, mineral gels such as aluminum hydroxide gel, oil-in-water emulsions, water-in-oil emulsions such as, e.g., Freund's complete and incomplete adjuvants, Block copolymer (CytRx, Atlanta Ga.), QS-21 (Cambridge Biotech Inc., Cambridge Mass.), SAF-M (Chiron, Emeryville Calif.), AMPHIGEN™. adjuvant, saponin, Quil A or other saponin fraction, monophosphoryl lipid A, and Avridine lipid-amine adjuvant. Non-limiting examples of oil-in-water emulsions useful in the vaccine of the invention include modified SEAM62 and SEAM 1/2 formulations. Modified SEAM62 is an oil-in-water emulsion containing 5% (v/v) squalene (Sigma), 1% (v/v) SPAN™ 85 detergent (ICI Surfactants), 0.7% (v/v) TWEEN™ 80 detergent (ICI Surfactants), 2.5% (v/v) ethanol, 200 pg/ml Quil A, 100 μg/ml cholesterol, and 0.5% (v/v) lecithin Modified SEAM 1/2 is an oil-in-water emulsion comprising 5% (v/v) squalene, 1% (v/v) SPAN™ 85 detergent, 0.7% (v/v) Tween 80 detergent, 2.5% (v/v) ethanol, 100 μg/ml Quil A, and 50 μg/ml cholesterol. Other immunomodulatory agents that can be included in the vaccine include, e.g., one or more interleukins, interferons, or other known cytokines.

The present invention also includes “variants” of the peptides, proteins, or amino acid sequences described herein.

For the purposes of the present invention, “variants” of an amino acid sequence comprise amino acid insertion variants, amino acid addition variants, amino acid deletion variants and/or amino acid substitution variants.

Amino acid insertion variants comprise insertions of single or two or more amino acids in a particular amino acid sequence. In the case of amino acid sequence variants having an insertion, one or more amino acid residues are inserted into a particular site in an amino acid sequence, although random insertion with appropriate screening of the resulting product is also possible.

Amino acid addition variants comprise amino- and/or carboxy-terminal fusions of one or more amino acids, such as 1, 2, 3, 5, 10, 20, 30, 50, or more segment of protein.

Amino acid deletion variants are characterized by the removal of one or more amino acids from the sequence, such as by removal of 1, 2, 3, 5, 10, 20, 30, 50, or more amino acids.

The deletions may be in any position of the protein. Amino acid deletion variants that comprise the deletion at the N-terminal and/or C-terminal end of the protein are also called N-terminal and/or C-terminal truncation variants.

“Antigen processing” refers to the degradation of an antigen into procession products, which are fragments of said antigen (e.g., the degradation of a protein into peptides) and the association of one or more of these fragments (e.g., via binding) with MHC molecules for presentation by cells, preferably antigen presenting cells to specific T cells.

“Coronavirus” as used herein refers to a genus in the family Coronaviridae, which family is in turn classified within the order Nidovirales. The coronaviruses are large, enveloped, positive-stranded RNA viruses. They have the largest genomes of all RNA viruses and replicate by a unique mechanism that results in a high frequency of recombination. The coronaviruses include antigenic groups I, II, and Ill. Nonlimiting examples of coronaviruses include SARS coronavirus including Covid-19, MERS coronavirus, transmissible gastroenteritis virus (TGEV), human respiratory coronavirus, porcine respiratory coronavirus, canine coronavirus, feline enteric coronavirus, feline infectious peritonitis virus, rabbit coronavirus, murine hepatitis virus, sialodacryoadenitis virus, porcine hemagglutinating encephalomyelitis virus, bovine coronavirus, avian infectious bronchitis virus, and turkey coronavirus, as well as chimeras of any of the foregoing (See Lai and Holmes “Coronaviridae: The Viruses and Their Replication” in Fields Virology, (4th Ed. 2001)).

A “coronavirus permissive cell” as used herein can be any cell in which a coronavirus can at least replicate, including both naturally occurring and recombinant cells. In some embodiments the permissive cell is also one that the nidovirus or coronavirus can infect. The permissive cell can be one that has been modified by recombinant means to produce a cell surface receptor in here ACE-2 for the SARS-CoV2.

The term “antigen” relates to an agent comprising an epitope against which an immune response is to be generated. The term “antigen” includes in particular peptides and proteins. The term “antigen” also includes agents, which become antigenic- and sensitizing-only through transformation (e.g. intermediately in the molecule or by completion with body protein). An antigen is preferably presentable by cells of the immune system such as antigen presenting cells like dendritic cells or macrophages. In addition, an antigen or a processing product thereof is preferably recognizable by a T or B cell receptor, or by an immunoglobulin molecule such as an antibody. In a preferred embodiment, the antigen is a disease-associated antigen, such as a tumor-associated antigen, a viral antigen, or a bacterial antigen.

In one embodiment, a disease-associated antigen is a tumor-associated antigen. In this embodiment, the present invention may be useful in treating cancer or cancer metastasis. In another aspect, this invention provides a method for treating or preventing cancer providing a vaccination protocol as a result of fusing a single or multiple tumor associated proteins showing different antigenic structure with the S2 Δ21 segment of SARS-CoV2-S protein, ensuring their expression in the cell and transporting them to the cell membrane with the help of S2 Δ21 and present there intensively to the immune system. Preferably, the diseased organ or tissue is characterized by diseased cells such as cancer cells expressing a disease-associated antigen and/or being characterized by association of a disease-associated antigen with their surface. Immunization with intact or substantially intact tumor-associated antigens or fragments thereof such as MHC class I and class II peptides or nucleic acids, in particular CM with antigen makes it possible to elicit a MHC class I and/or a class II type response and, thus, stimulate T cells such as CD830 cytotoxic T lymphocytes which are capable of lysing cancer cells and/or CD4+ T cells. Such immunization may also elicit a humoral immune response (B cell response) resulting in the production of antibodies against the tumor-associated antigen. In one embodiment, the term “tumor-associated antigen” refers to a constituent of cancer cells which may be derived from the cytoplasm, the CM and the cell nucleus. In particular, it refers to those antigens which are produced, preferably in large quantity, intracellularly or as CM antigens on tumor cells.

The term “treat” or “treatment” relates to any treatment which improves the health status and/or prolongs (increases) the lifespan of an individual. Said treatment may eliminate the disease in an individual, arrest or slow the development of a disease in an individual, inhibit or slow the development of a disease in an individual, decrease the frequency or severity of symptoms in an individual, and/or decrease the recurrence in an individual who currently has or who previously has had a disease.

In particular, the term “treatment of a disease” includes curing, shortening the duration, ameliorating, slowing down or inhibiting progression or worsening of a disease or the symptoms thereof.

The term “immunotherapy” relates to a treatment preferably involving a specific immune reaction and/or immune effector function(s).

The term “immunization” or “vaccination” describes the process of treating a subject for therapeutic or prophylactic reasons.

A vaccine is a biological preparation that provides active acquired immunity to a particular infectious disease. A vaccine typically contains an agent that resembles a disease-causing microorganism and is often made from weakened or killed forms of the microbe, its toxins, or one of its surface proteins. A vaccine can be generated using recombinant DNA technology is called recombinant vaccine. Recombinant vaccines can be created in a number of different methods. These types of vaccines are manufactured with the assistance of expression systems, such as bacteria, insect, yeast, plant, mammalian, and cell-free. While there are various types of vaccines made possible by recombinant DNA technology, recombinant vaccines can be classified into two major categories. DNA vaccines usually consist of synthetic DNA containing the gene that encodes the disease-agent protein. Usually, the plasmid DNA used as vaccine is propagated in bacteria such as E. coli and they are isolated and purified for injection. This “naked” DNA is usually injected intramuscularly or intradermally. The principle behind a DNA vaccine is that the antigen can be expressed directly by host cells in a way that simulates viral infection and invokes an immune response from the host. Recombinant (protein subunit) Vaccines are subunit vaccines containing only a fraction of the pathogenic organism. Often time these are synthetic peptides that represent the protein component that induces an immune response. But they can also consist of protein subunits (antigens) expressed in a heterologous expression system (E. coli, yeast, insect etc.) using recombinant protein expression technologies. Prokaryotic expression systems for vaccine antigen production include bacteria such as E. coli and eukaryotic systems include mammalian, yeast or insect cells. Several factors are taken into consideration before selecting the right system for vaccine antigen expression. Among other things, expression levels, selection marker and the presence or absence of post-translational modification are essential factors that interfere in the efficacy of production of recombinant antigens as vaccines.

Therefore, the present invention provides a new antigen expression and presenting system which provide protein (antigen) in large amounts and in its natural structure in the eucaryotic cell membrane and its use as a whole cell or CM will be a new vaccination protocol.

The present invention demonstrated that presenting the S1 of SARS-CoV2 in large amount and in its natural structure in CM will provide a much stronger and more specific immune response.

Most importantly, in this way it will be possible to prepare an almost unlimited amount of SARS-CoV2-S antigen in its native form using the cell lines in a short time.

In addition, since the S protein will be attached to the cell membrane, it will not be able to circulate freely, so it will not pass the blood-brain barrier and it may not show the cell signal stimulation effects mentioned above.

The present invention further provides, in another aspect, a method for treating or preventing viral infection, providing a vaccination protocol as a result of fusing multiple viral proteins (more than one in the same cell membrane) showing different antigenic structures with the SARS-CoV2-S2 protein, ensuring their expression in the cell and transporting them to the cell membrane with the help of SARS-CoV2-S2 protein and presents there intensively to the immune system.

This process can be applied by fusing different antigenic proteins or peptides to the SARS-CoV2-S2 protein in a single vector system in frame shift. This process also can be achived in the same cell using vector systems with different selection markers to express each antigenic protein fused to SARS-CoV2-S2 protein.

The agent stimulates the body's immune system to recognize the agent as a threat, destroy it, and to further recognize and destroy any of the microorganisms associated with that agent that it may encounter in the future. Vaccines can be prophylactic (to prevent or ameliorate the effects of a future infection by a natural or “wild” pathogen), or therapeutic (to fight a disease that has already occurred, such as cancer).

The administration of vaccines is called vaccination. Vaccination is the most effective method of preventing infectious diseases; widespread immunity due to vaccination is largely responsible for the worldwide eradication of smallpox and the restriction of diseases such as polio, measles, and tetanus from much of the world.

The term “carrier” refers to an organic cell or CM, of a natural, with which the active component is combined in order to facilitate, enhance or enable application.

As described in detail, CM and/or whole cell was used as the carriere in this invention.

The present invention is described in detail by the following figures and examples which should be construed by way of illustration only and not by way of limitation. On the basis of the description and the examples, further embodiments are accessible to the skilled worker and are likewise within the scope of the invention.

### EXAMPLES

### Confirmation of Cell Membrane Isolation

Cell membrane and cytoplasm prepared according to the specified protocol in material and method were placed on the nitrocellulose membrane in equal amounts and then investigated by dot blot system using antibodies specific to the proteins in the cell membrane or cytoplasm. Cog2 (Conserved oligomeric Golgi complex subunit 2), required for proper Golgi morphology and function, is a 730 amino acid component of the COG complex and is localized to the cytoplasmic side of the Golgi apparatus. Calreticulin and calnexin are nascent proteins related to the endoplasmic reticulum. They both are also integral proteins and found in the cell membrane different amount depending on cell type.

Glyceraldehyde-3-phosphate dehydrogenase (GAPDH) is an enzyme related to glycolysis and is mainly found in the cytoplasm. However, GAPDH has been shown to be present in the membrane, albeit in small amounts. FIG. 1-A. shows that while both GAPDH and Cog2 are almost absent in the membrane and found in the cytoplasm, Calreticulin and calnexin are found in both the membrane and cytoplasm, as expected.

### SARS-CoV2 Spike Δ21 Protein Overexpression Places Spike Protein in the Cell Membrane

As previously described when ER retrieval signal in its cytoplasmic tail of S protein mutated allows transport of the full-length S protein to the plasma membrane. Therefore, we investigated expression of spike protein on cell membrane of cells expressing SARS-CoV2 Spike wt or SARS-CoV2 Spike Δ21 using dot blot assay. The culture plates were seeded with in 1×106 of TA-Hep3B containing pRetroX-Tight-Pur-hSARS-CoV2-S wt, or pRetroX-Tight-Pur-hSARS-CoV2-S Δ21, and TA-Renca and TA-MAD109 cells containing pRetroX-Tight-Pur-mSARS-CoV2-S wt or pRetroX-Tight-Pur-mSARS-CoV2-S Δ21, doxacillin (dox) was added after 24 hours after seeding the cells. Then, after 12, 24 and 48 hours, the cells were prepared and used in the different studies. FIGS. 1b-e show the dotblot results, in which the cells expressing Spike Δ21, almost all of the spike protein was found in the cell membrane, whereas in cells expressing spike wt, no spike protein was detected in the cell membrane in both Hep3B and Renca cells. In addition, as a result of test with IHC (without permeabilization process), SARS-CoV2 S protein was determined intensly in the cells expressing SARS-CoV2 S Δ21, while it was not determined in cells expressing SARS-CoV2 S wt (FIG. 1f-g). FIGS. 1h-j show the amount of SARS-CoV2 S protein after induction with dox in TA-Hep3B containing pRetroX-Tight-Pur-hSARS-CoV2-S Δ21, and TA-Renca and TA-MAD109 cells containing pRetroX-Tight-Pur-mSARS-CoV2-S Δ21 cells over time.

### Cell Fusion Occurs in the Presence of S Δ21Expression

Hep3B cell containing pRetroX-Tight-Pur-S wt or pRetroX-Tight-Pur-S Δ21 were split into 24-well plates, 3×105 cell per well. After 24 hours, the medium in the wells was removed and replaced with medium with dox. Twenty-four hours after dox addition, cells were stained with hoechst (1 ug/ml) or IHC stained using anti-SARS-CoV2-S antibody and viewed with a flourescence microscope. Cell-cell fusion was observed in Hep3B cell containing pRetroX-Tight-Pur-S Δ21 24 hours after dox adding (FIGS. 2a and b), while in 48 hours advanced unification of the cells was observed to distrupt the cell structure (FIG. 1h). Although, not as dramatic as in Hep3B cells, S Δ21 expression also creates a fusion in Renca and MAD109 cells (FIGS. 1i and J).

### SARS-CoV2 Spike Δ21 Expressing Cell Provides a Large Amount of Spike Protein Expression in the Membrane of Cell Determined by ELISA Assay.

In the next experiment, The wells of 96 cell culture plates were seeded with in the range of 1×101 to 1×104 of TA-Hep3B, TA-Renca and TA-MAD109 cells containing pRetroX-Tight-Pur-hSARS-CoV2-S wt, pRetroX-Tight-Pur-hSARS-CoV2-S Δ21, and pRetroX-Tight-Pur-mSARS-CoV2-S wt and pRetroX-Tight-Pur-mSARS-CoV2-S Δ21, dox was added after 24 hours and fixed with 4% paraformalhdehyte after 12, 24 and 48 hours. The total levels of IgG, anti-SARS-CoV2-S antibodies of the serum of the person in whom a high amount of anti-SARS-CoV2-S antibodies detected with commercial ELISA assay were measured using the ELISA protocol. Here, the ELISA test protocol was completed without permeabilization. ELISA test shows that the signal in increases in accordance with the amount of cells expressing SARS-CoV2-S Δ21 and the duration of dox administration (FIGS. 3a and b). However, in the ELISA test cells expressing SARS-CoV2-S wt, almost no signal was detected in Renca (FIG. 3c) and Hep3B cell lines (FIG. 3d). ELISA test shows that the signal in increases in accordance with the amount of cells expressing SARS-CoV2-S Δ21 when the anti-S1 antibody (Biolegend) used (FIG. 3d). In addition, it was determined that permeabilization process did not increase the amount of signal in the cells expressing SARS-CoV2-S Δ21 (data was not shown).

### SARS-CoV2-S Δ21 Replaces Other Cell Membrane Proteins

Next, we asked the question of how high levels of S protein in the cell membrane affects other membrane proteins. In other words, how the cell membrane can handle such a load of S protein and how is it possible?

For this purpose, we investigated the amounts of the two basic proteins that make up the structure of the cell membrane including Spectrin Alpha Erythrocyticl (SPTA1) which is a member of a family of molecular scaffold proteins that link the plasma membrane to the actin cytoskeleton and functions in the determination of cell shape and N-cadherin (CDH2) which is a transmembrane protein expressed in multiple tissues were investigated in the cell membrane and cytoplasm in cells with SARS-CoV2-S S Δ21 or without SARS-CoV2-S S Δ21 expression.

Cell membranes and cytoplasms of rtT expressing TA-Hep3B (control cell), and TA-Hep3B containing pRetroX-Tight-Pur-hSARS-CoV2-S Δ21 and TA-Renca (control cell), and TA-Renca containing pRetroX-Tight-Pur-mSARS-CoV2-S Δ21 were isolated after 36 hours of treatment with dox and amount of SPTA1 and CDH2 in the cell membrane and cytoplasm were investigated by dotblot using SPTA1 or CDH2 specific antibodies.

FIGS. 4a-d show that SPTA1 and CDH2 proteins are 2-3 times less in the cell membrane of both Hep3B and Renca cells expressing SΔ21 compared to the Hep3B and Renca cells not expressing SΔ21. Therefore, as a result of SARS-CoV2-S Δ21 overexpression in the cell, S1 protein is transported to the cell membrane and replaces the major membrane proteins SPTA1 and CDH2. In addition to confirm the dot blot results, after dox addition, SARS-CoV2-S protein expression (FIG. 4e) and the amount of cell membrane proteins (SPTA1 and CDH2) were examined by ELISA at 0, 12, 24 and 48 hours (FIGS. 4f and g). As seen in the FIGS. 4f and g, it was determined that the cell membrane proteins including SPTA1 or CDH2 decreased in the cell membrane at a rate similar to the amount of increase of S protein (FIG. 4e) in the cell membrane. In addition, the same result was obtained in the ELISA test using SARS-CoV2-S negative patient serum as source of the autoantibody against to the cell membrane. It was determined that the cell membrane proteins that bind to human serum antibodies decreased in the cell membrane at a rate similar to the amount of increase of S protein (FIG. 4h).

### Immunization of Mice

Balb/c and C57BL/6 mice groups were immunized subcutenously (SC) with 5×104 TA-Renca-mSARS-CoV2-S Δ21 whole cell (WhC) (3 animals/group) or cell membrane (CM) obtained from the 5×104 TA-Renca-mSARS-CoV2-S Δ21 cell together with and without complete freund's adjuvants (CFA) in the A. U. Medical School Experimental Animal Care Unit (4 animals were used for each group). The cells and cell membranes were obtained 36 hours after the adding dox on growing cell culture. Furthermore, 4 animals/group were immunized with 5×104 TA-Renca-mSARS-CoV2-S Δ21 cell or cell membrane obtained from the 5×104 TA-Renca-mSARS-CoV2-S Δ21 cell line without CFA and 4 animals/group were immunized with PBS+CFA as plasebo. At the same time periods, 4 mice were immunized with the defined Covid-19 mRNA S vaccina (BNT162b2) administered at the dose of ml/kg described for adult humans and the other 4 groups of mice were immunized with the whole inactivated virus (WIV) (Sinovac-Coronavac Covid-19) administered at the dose of ml/kg described for adult humans. Blood samples were collected from the tail vein on days 15 and 28 and were used in measuring antibody titer and virus neutralization assay.

### Antibody Measurements by ELISA in Immunized Mice

The total levels of IgG, anti-SARS-CoV2-S antibodies in individual immunized mice were measured by ELISA. Blood samples were collected from the tail vein on days 15 and 28 and were used in measuring antibody titer and virus neutralization assay.

As a result, the mean endpoint titers of S protein specific IgG in mice immunized with a single vaccination of the Renca WhC expressing SARS-CoV2-S (WhC-S) with CFA was significantly higher than those observed in mice received PBS with CFA in Balb/C (FIG. 5a) and C57BL/6 (FIG. 5b). Additionally, S protein specific IgG in mice immunized with the CM expressing SARS-CoV2-S(CM-S), although not significant, a slight increase was observed compared to the goup of mice immunized with the WhC-S (FIG. 5c). In addition, adding CFA to immunization, although not significant, slightly increased S protein specific IgG in both WhC-S and CM-S immunizations (FIG. 5d).

It was determined that vaccination with CM-S produced a statistically stronger immune response than vaccination with BNT162b2 (FIG. 5e). It was also determined that the difference between these two groups of vaccination systems was higher in single dose vaccination (FIG. 5e). In addition, in comparison with the pool of human convalescent patient serum (hcPS), vaccination with CM-S produced a statistically significant stronger immune respons even using a single dose of CM-S vaccination (FIG. 5e).

In the both neutralization tests with cell presenting SARS-CoV2-S protein (WhC-S) and NM coated with RBD unit of SARS-CoV2-S protein, a statistically significant difference was observed in the double vaccination with CM-S compared to the serum of person double-vaccinated with BNT162b2 (mRNA-S—PS), the serum of the mice group double-vaccinated with BNT162b2 (mRNA-S), and hcPS (FIGS. 6a and b). All the neutralization tests were completed with sera obtained after the booster dose.

### Live SARSCoV-2 Neutralization Assays

Similar to the neutralization tests described above, in Live SARSCoV-2 neutralization assays, a statistically significant difference was observed in the double vaccination of mice with CM-S compared to the mice group double-vaccinated with BNT162b2 (mRNA-S), human double-vaccinated with BNT162b2 (mRNA-S—PS), and hcPS (FIG. 6c).

The mean NT50 (50% neutralization titers) value in the CM-S was 8,2-, 3,7- and 4,5-fold higher than those observed in the serums of mice group double-vaccinated with BNT162b2 (mRNA-S), human double-vaccinated with BNT162b2 (mRNA-S—PS) and the hcPS, respectively (FIG. 6c).

### Immune Response Type Assessment

As indicated, multiple SARS-CoV-2 vaccine types, such as DNA-, RNA-based formulations, recombinant-subunits containing viral epitopes, adenovirus-based vectors and purified inactivated virus are developed. To fully assess the immunogenicity and safety of multiple SARS-CoV-2 vaccine antigens, the preclinical animal model testing is necessary. In humans, the induced virus neutralization ability is the gold standard for determining vaccine efficacy. But in mouse vaccine assessment model, the detection of IgG, IgG1 & IgG2a antibody titer and IgG1-IgG2a ratio is a better correlate for vaccine efficacy than neutralization alone. In-depth understanding of the immune response in animal model will help to assess the protective capability, toxicity and safety of the SARS-CoV-2 vaccines.

The increased IgG1/IgG2a ratio indicates the Th2 type humoral immune response, the increased IgG2a/IgG1 ratio tends to Th1 type cellular immune response. The balance of Th1/Th2 immune response will help improve the protective effect of the vaccine and avoid pathological enhancement after immunity. Increased induction of both antibody isotypes as measured by ELISA was a better correlate for vaccine efficacy than neutralization alone.

FIG. 6d shows that the CM-S elicited 4,2 and 4,9-fold more IgG1 than the mRNA-S and WIV vaccinated mice, respectively and CM-S also elicited 4,3 and 3,2-fold more IgG2a than the mRNA-S and WIV vaccinated mice, respectively. Interestingly, although WIV's neutralizing antibody titers are lower than that of mRNA-S vaccination (data not shown), it secretes more IgG2a compared to mRNA-S vaccination. In addition, while IgG2a/IgG1 ratio changed in favor of the Th2 type humoral immune response in CM-S and mRNA-S vaccinated animals, it changed in favor of Th1 type cellular immune response in the WIV vaccinated mice (FIG. 6d).

### Material and Methods

### Cells

Hep3B human hepatocelluar carcinoma cell line, Renca cell lines derived renal cortical adenocarcinoma in Balb/c mice, MAD109 mice lung cancer cell lines were used in these experiments. The cells were grown in DMEM or RPMI mediums supplemented with 10% FBS, 1% Pen/Strep, 1% L-glutamine. All cells were incubated at 37° C., 5% CO2 conditions.

Mouse Codon Optimized SARS-CoV2-S-Wt and -Δ21 cDNA Preparation

Mouse codon optimized SARS-CoV2-S wt and Δ21 cDNA preparation

SARS-CoV2 strain Whuan-Hu-1(Genebank NC 045512) spike genome was codon-optimized to mouse (mARS-CoV-2 S wt) and prepared as mARS-CoV-2 S wt and mARS-CoV-2 S Δ21 by GeneScript (https://www.genscript.com).

Codon optimized to H. sapiens SARS-CoV2-S-wt was a gift from Gerald Pao (Addgene plasmid #141347; http://n2t.net/addgene:141347; RRID:Addgene_141347). Codon optimized to H. sapiens SARS-CoV2-S-Δ21 was a gift from Jesse Bloom (Addgene plasmid #155130; http://n2t.net/addgene:155130; RRID:Addgene_155130).

### Overexpression of SARS-CoV2-S Wt and -SΔ21 Using the Retro-X Tet-on Advanced Inducible Expression System in Cells

The Retro-X Tet-On Advanced Inducible Expression System requires the simultaneous presence of two retroviral constructs in a cell, Tetracyclin dependent rtTA transactivator (pRetroX-Tet-On-Advanced) and the target construct driven by the transactivator (pRetroX-Tight-Pur plus target gene). Thus, we first established the rtTA transgene expressing, Hep3B human orginated cell line and Renca and MAD109 mouse cells lines. For this purpose, Hek293T cells were transfected by the pRetroX-Tet-On-Advanced and helper vectors pCMV-VSV-G, pUMVC, using CalPhos Mammalian Transfection Kit (631312-Takara bio). After 48 h supernatants containing rtTA virus were collected and target cells were infected under the presence of 10 mg/ml polybrene (TR-1001, Sigma-Aldrich). After 24 hours, the media of the cells were replaced with media containing neomycin (G418-RO, Sigma-Aldrich) 0.2-1 mg/ml and followed 10 to 15 days for selection. Then living cells were combined in a pool and named as TA-Hep3B, TA-Renca and TA-MAD109

Cloning of Codon Optimized for Human SARS-CoV2-S Wt and Δ21 (hSARS-CoV2-S and Δ21) and Codon Optimized for Mouse SARS-CoV2-S Wt and -SΔ21 (mSARS-CoV2-S and -SΔ21 Genes into Modified pBSK (pBSK-clo2D) Subcloning Vector Plasmids.

The hSARS-CoV2-S wt, hSARS-CoV2-S Δ21, mSARS-CoV2-S wt, mSARS-CoV2-S Δ21 genes were inserted into the pBSK-clo2D vector construct, which was previously prepared the cloning sites of the pBSK (−) vector as KpnI, ApaI, XhoI, SaII, PmeI, BamHI EcoRV, EcoRI, XbaI, NotI, HindIII, MfeI, EcoRI, PstI, SmaI, BamHI, XbaI, BstXI, and SacI in order to facilitate general DNA cloning in my laboratory. Blunt ended SARS-CoV2-S genes were inserted into the pBSK-clo2D plasmid cut with EcoRV and dephosphorylated with dephosphorylation enzyme (FastAp thermosensitive Alkaline Phosphatase (Thermoscientific-ThermoFisher USA). pBSK-hSARS-CoV2-S wt, pBSK-hSARS-CoV2-S Δ21, and pBSK-mSARS-CoV2-S wt and pBSK-mSARS-CoV2-S Δ21 vectors were obteined. Sequence analysis for the mutation of the each vector was searched.

Cloning of Codon Optimized for Human hSARS-CoV2-S Wt and Δ21, and for Mouse mSARS-CoV2-Swt and Δ21 Genes into pRetroX-Tight-Pur Vector.

hSARS-CoV2-S wt and S Δ21, and mSARS-CoV2-S wt and S Δ21 genes were obtained from the pBSK-hSARS-CoV2-S wt, pBSK-hSARS-CoV2-S Δ21, and pBSK-mSARS-CoV2-S wt and pBSK-mSARS-CoV2-S Δ21 vectors respectively by cutting with with BamHI enzyme. pRetroX-Tight-Pur retroviral vector was cut by BamHI restriction enzyme and dephospharylated and ligated with hSARS-CoV2-S wt and SΔ21, and mSARS-CoV2-S wt and SΔ21 cDNAs with compatible cohasive end. After the oriantation construct was determined, pRetroX-Tight-Pur-hSARS-CoV2-S wt, pRetroX-Tight-Pur-hSARS-CoV2-S Δ21, and pRetroX-Tight-Pur-mSARS-CoV2-S wt and pRetroX-Tight-Pur-mSARS-CoV2-S Δ21 vectors were reproduced in large volumes using DH5a E. Coli.

In the next step, Hek293T cells were transfected by the pRetroX-Tight-Pur-hSARS-CoV2-S wt, pRetroX-Tight-Pur-hSARS-CoV2-S Δ21, and pRetroX-Tight-Pur-mSARS-CoV2-S wt and pRetroX-Tight-Pur-mSARS-CoV2-S Δ21 vectors and helper vectors pCMV-VSV-G, pUMVC, using CalPhos Mammalian Transfection Kit (631312-Takara bio) separately.

After 48 h supernatants of pRetroX-Tight-Pur-hSARS-CoV2-S wt, pRetroX-Tight-Pur-hSARS-CoV2-S Δ21, and pRetroX-Tight-Pur-mSARS-CoV2-S wt and pRetroX-Tight-Pur-mSARS-CoV2-S Δ21 vectors containing viruses with medium from the individual cells were collected and rtTA expressing TA-Hep3B, TA-Renca and TA-MAD109 target cells were infected. The infected clones were selected by 0.5-3 mg/ml puromycin (P9620-Sigma-Aldrich) for 6 days. Infected clones were maintained in the presence of 250 ng/ml puromycin. The expresssion of SARS-CoV2-S wt and SARS-CoV2-S Δ21 in individual cell lines was followed by dotblot and immunohystochemistry (IHC).

### Overexpression of SARS-CoV2-RBD Using the Retro-X Tet-on Advanced Inducible Expression System

For this purpose, the mouse RBD and human RBD were PCR amplified from the mSARS-CoV2-S Δ21 or hSARS-CoV2-S Δ21 cDNAs using the sense primer containing kozak sequence and the anti-sense primer containing stop codon mRBD-F 5′-GCCGCCGCCATGCAGCCAAACCGAGTCC-3′, mRBD-R 5′-TTAGAAGTTCACGCACTTGTTC-3′, hRBD-F 5′-GCCGCCGCCATGCAACCCACCGAGTCCATTGTG-3′ and hRBD-R 5′-TTAATTTACGCACTTGTTCTTC3′ respectively. It was then cloned into pRetroX-Tight-Pur vector as described above and inserted into cells for use in the dox inducible system.

### Isolation of Cell Membrane.

The membrane was isolated from cells as previously described with some modifications (Zhai et al. 2017, Cao, et al. 2016). In brief, cells were harvested and resuspended at a concentration of 2.5×107 cells/mL in ice-cold Tris-magnesium buffer (TM buffer, pH 7.4, 0.01 M Tris and 0.001 M MgCl) and homogenized with dounce homogenizer to disrupt the cells. The cell homogenate was mixed with 1 M sucrose to a final concentration of 0.25 M sucrose, and then centrifuged at 2000 g and 4° C. for 10 min. The supernatant was collected and further centrifuged at 3000 g and 4° C. for 30 min to collect the cell membrane. The cell membrane was washed with ice-cold TM-buffer with 0.25 M of sucrose and collected by centrifugation at 3000 g and 4° C. for 30 min.

### Dot Blot

Cell membrane-containing solution (also cytoplasmic extract) was diluted with PBS (137 mmol/L NaCl, 2.7 mol/L KCl, 10 mol/L Na2HPO4, and 1.8 mmol/L KH2PO4, pH 7.4) to 1×103 cells in 5 ul and applied onto on a nitrocellulose membrane (NM). Then the dots were allowed to dry. The NM was completely immersed in blocking solution (5% non-fat skim milk in PBS) and then incubated for 60 min. It was washed three times by complete immersion in PBS-tween (PBS with 0.05% tween 20) for 1 min every time. The NM was immersed in 1/100 diluted with antibodies or serum containing anti-SARS-Cov2S antibody in PBS-milk (5% non-fat skim milk in PBS) and incubated for one hour. The NM was washed three times with PBS-tween as described above. Depending on the primary anibody used, the NM was completely immersed with anti-human, anti-mouse or anti-rabbit IgG antibody (Ab) conjugated with horseradish peroxidase (Thermo Fisher Scientific, USA) diluted 1/1500 in PBS-milk, and incubated for 45 min. It was washed three times again with PBS-tween. Finally, the blot reaction was revealed by immersing the membrane completely in the HRP chemiluminescence for 5 min and visualized by Bio-Rad chemiDoc system. Each NM was analyzed in duplicate. Intensity of dots was investigated by Imagej (https://image.nih.gov./ij) program.

### Whole Cell ELISA Preparation

The wells of 96 cell culture plates were seeded with 1×103 of TA-Hep3B, TA-Renca and TA-MAD109 cells containing pRetroX-Tight-Pur-hSARS-CoV2-S wt, pRetroX-Tight-Pur-hSARS-CoV2-S Δ21, and pRetroX-Tight-Pur-mSARS-CoV2-S wt and pRetroX-Tight-Pur-mSARS-CoV2-S Δ21, doxacillin (dox) was added after 24 hours and fixed with 4% paraformalhdehyte after 36 hours.

### Cell Membrane ELISA Preparation

The cell membranes containing S protein were prepared 36 hours after the dox adding into the cell according to the protocol described above. TM buffer containing Cell membrane was diluted with coating buffer (05M carbonate-bicarbonate buffer, pH 9.6) and used to coat all 96 wells of ELISA plates to 1×103 cells per well.

ELISA Preparation with S1 and RBD Subunits of SARS-CoV2 s

ELISA plates were coated overnight with 20 ng/ml of SARS-CoV2 S1 (Biolegend, USA), or RBD obtained from the cell decribed above (SARS-CoV2-S RBD expressing cell) was diluted with coating buffer and used to coat all 96 wells of ELISA plates.

### Antibody Measurements Using Whole Cell or Cell Membrane Coated ELISA.

The total levels of IgG, anti-SARS-CoV2-S antibodies in individual persons who have been infected or vaccinated were measured by prepared ELISAs described above. Ninety-six-well cell culture plates with cell containing hSARS-CoV2-Swt, hSARS-CoV2-S Δ21, mSARS-CoV2-Swt and mSARS-CoV2-S Δ21 protein on cell membrane described above or coated with cell membrane containing covid-19 S protein were blocked with 5% milk in PBS for 1.5 h at room temperature (RT).

Serum samples from each immunization group were diluted in PBS-Tween-1% milk, added to plates, and incubated for 1 h at RT. Plates were then washed, and secondary HRP-conjugated anti-human IgG, antibodies (Solarbio-China; all diluted 1:1,000 in PBS-Tween-1% milk) were added and incubated for 45 min at RT. Plates were washed, the o-phenylenediamine dihydrochloride (OPD) substrate (Sigma-Aldrich-0.5-1.0 mg/ml OPD was dissolved in buffer −0.05M citric acid, 0.05M sodium phosphate; pH 5) and then 1 ul %30 hydrogen peroxide was added per 1 ml of substrate) was added, and the reaction was stopped by adding 1 M H2SO4. Absorbance was read at 450 nm.

### Expression Analysis of SARS-CoV2-S by Immunofluorescence Microscopy.

Immunofluorescence experiments were performed in SARS-CoV2-S Δ21 expressing Hep3B, Renca and MAD109 cells. Twent-fours after splitting the cells, dox added into the cell except to control cells. The cells permeabilized or non-permeabilized cells were incubated with SARS-CoV2-S pozitif serum of patient infected with SARS-CoV2-S or anti-S antibody (Biolegend) after fixation and blocking at 12, 24 and 48 hours after dox adding. Anti-SARS-CoV2-S was detected with a rabbit or human secondary antibodies conjugated with the FITC fluorochrome (Solarbio; diluted 1:1000). We used Hoecst (Sigma) to stain the cell nuclei. Images of sections of the cells were taken with a flouresence microscope.

### Ethics Statement.

Mice studies were carried out at Ankara University Medical School Experimental Animal Care Unit under permission of the Local Ethics Committee for Animal Experiments at Ankara University with the supervision of a veterinarian ((File No:2021-54; permit number: 2021-1074). The animal protocols were approved by the Local Ethics Committee for Animal Experiments at Ankara University (A.U.). All animal experiments and methods were performed in accordance with the animal experimentation guidelines and regulations approved by the Local Ethics Committee for Animal Experiments at A.U. The study was carried out in compliance with the ARRIVE guidelines. Live virus studies were completed in the Generel Directorate of Public Health, Microbiology Reference Laboratory (Animal Biosafety Level 3 plus—ABSL3β) in Ankara-Turkey.

### Animal Immunization

Balb/c and C57BL/6 mice groups (4 animal for each group) were immunized subcutenously with 5×10⁴ whole cell expressing TA-Renca-mSARS-CoV2-S in a volume of 100 ul or cell membrane obtained from the 1×10⁴ TA-Renca-mSARS-CoV2-S Δ21 expressing cell in a volume of 100 ul in the A. U. Medical School Experimental Animal Care Unit. The cells and cell membranes were obtained 36 hours after the adding dox on growing cell culture. Cell membrane isolated according to the protocol decribed above, and cell collected with scraped and washed 3 times with PBS were given to mice with complete freund's adjuvants (CFA, Sigma-Aldrich) at 1st and 15th days. In the second vaccination incomplete freund's adjuvants was used. IgG titers were determined by ELISA in sera collected on day 15 or 28 post vaccination from mice immunized with cell or cell membrane containing mSARS-CoV2-S Δ21. Control group received PBS with CFA or the same amount of cell membrane of Renca wt cell. Furthermore, 4 animals/group were immunized with 1×10⁴ TA-Renca-mSARS-CoV2-S Δ21 cell or cell membrane obtained from the 1×10⁴ TA-Renca-mSARS-CoV2-S Δ21 cell line without CFA and 4 animals/group were immunized with PBS+CFA as plasebo. At the same time periods, 4 mice were immunized with the defined Covid-19 mRNA S vaccine (BNT162b2) administered at the dose of ml/kg described for adult humans and the other 4 groups of mice were immunized with the inactive whole virus vaccine (Sinovac-Coronavac Covid-19) administered at the dose of ml/kg described for adult humans. Blood samples were collected from the tail vein on days 15 and 28 and were used in measuring antibody titer and virus neutralization assay.

Blood from each individual mouse was collected and processed to obtain serum samples to analyze the titers of IgG antibodies, IgG isotypes, and neutralizing antibodies against SARS-CoV-2.

### Immunogenicity Evaluation of Vaccinations

To detect antibody levels in the sera of mice vaccinated with cell or cell membrane containing mSARS-CoV2-S Δ21, geometric dilutions of the sera for ELISA were used for the immunogenicity analyses. Endpoint titer referred to reciprocal serum dilutions that gave a mean OD value three times greater than the pre-immune control samples.

### Enzyme-Linked Immunosorbent Assay.

The total levels of IgG, anti-SARS-CoV2-S antibodies in individual immunized mice were measured by ELISA. Ninety-six-well cell culture plates with cell containing mSARS-CoV2-S were blocked with 5% milk in PBS for 2 h at room temperature (RT).

To detect antibody levels in the sera of mice vaccinated with cell or cell membrane containing mSARS-CoV2-S Δ21, twofold serial dilutions of the sera for ELISA were used for the immunogenicity analyses. Serum samples from each immunization group were diluted in PBS-Tween-1% milk, added to plates, and incubated for 1 h at RT. Plates were then washed, and secondary HRP-conjugated anti-mouse IgG, antibodies (Biolegend; all diluted 1:1,000 in PBS-Tween-1% milk) were added and incubated for 45 min at RT. Plates were washed, the OPD substrate (Sigma-Aldrich) was added, and the reaction was stopped by adding 1 M H2SO4. Absorbance was read at 450 nm. Total IgG titers were measured as the last dilution that gives an absorbance at least three times higher the absorbance of a naive serum.

### Neutralization Assay.

The capacity of the sera obtained from mice immunized with vaccine candidates TA-Renca-mSARS-CoV2-S Δ21 cell membrane (CM) to neutralize SARS-CoV2 virus was initially determined by two different protocols. In the first protocol, whole cell obtained from the 1×10⁶ TA-Renca-mSARS-CoV2-S Δ21 cell after 36 hours dox induction (total 1 ml PBS) was used for the neutralization assay after 3 times washing with PBS. 100 ul of cell membrane was incubated with twofold serially diluted mouse sera for 1 h at 37° C. The mixtures were then centrifuged and supernatant used in ELISA with RBD coated 96 plates and relative neutralization titer was determined. The ID50 was determined as the highest dilution of serum which resulted in a 50% reduction of OD450 signal in ELISA in comparison with negative serum control.

In the second protocol, cytoplasmic proteins containing RBD unit of SARS-CoV2-S, which is overexpressed in the TA-Renca under dox control, were adsorbed into the NM (nitrocellulose membrane). For the neutralization assay, NM with RBD was incubated with twofold serially diluted mouse sera for 1 h at 37° C. The mixtures were then centrifuged (antibodies bound to RBD are removed-neutralized) and supernatant used in ELISA on 96 well plate containing SARS-CoV2-S antigen.

### Live SARS-CoV2 Neutralization Assay.

Presence of virus specific neutralizing antibodies in the mice vaccinated with CM-S, mRNA-S and WIV were evaluated via virus neutralization assay. The serially-diluted (two-fold in DMEM) serum samples were mixed with an equal volume of 100TCID50 virus (1:10000) titer in duplicate, and incubated for 1 h at 370 C. The serum-virus mixtures were subsequently inoculated onto one day-old 90% confluent Vero E6 cells and were grown in 96-well plates. The infected cells were further incubated under the identical conditions for four days. The test was evaluated via inverted microscope when 100% CPE was observed in virus control wells.

### IHC

Cells were fixed in buffered formalin (4%) solution. Purified SARS-CoV2 IgG from convalescent human plasma or anti-SARS-CoV2 S1 antibody (Biolegend) was used as primary antibody, which was followed by incubation with FITC labelled anti-human IgG secondary antibody (Sigma Aldrich, USA) for an hour. The cells were finally visualized flourescence microscope on a slide.

### Statistical Analysis.

All of the data are expressed as the means t standard errors of the means. For all of the analyses, P values were obtained from Student's t-test (unpaired, two tailed) or Spearman rank-correlation tests. All of the graphs were generated using Microsoft Excel. The data are based on the results of at least three independent experiments. The error bars show the standard deviations.

### BRIEF DESCRIPTION OF THE SEVERAL VIEWS OF THE DRAWINGS

FIG. 1 SARS-CoV2 S Δ21 expression ensures that Spike protein is located in the cell membrane. a. Confirmation of cell membrane (CM) isolation. b, c. SARS-CoV2 S Δ21 expression localizes the entire spike protein to the Hep3B CM and the Renca CM shown by dotblot, respectively. d, e. SARS-CoV2 S WT expression localizes the entire spike protein to the Hep3B and the Renca cells' cytoplasm, as shown by dotblot respectively. f, g. While SARS-CoV2 S Δ21 expression localizes the entire spike protein to the Hep3B CM and the Renca CM this does not occur in cell with SARS-CoV2 S wt expression, as shown by IHC. h, i, and j. SARS-CoV2 S Δ21 expression localizes the entire spike protein to the Hep3B CM, the Renca CM and MAD109 cell line membrane shown by IHC. After induction of cells containing pRetroX-Tight-Pur-SARS-CoV2-S Δ21 with dox, spike protein expression at different time intervals was shown by IHC.

FIG. 2 Cell fusion occurs in the presence of SARS-CoV2-S Δ21 expression. Twenty-four hours after dox addition, Hep3B, cells containing pRetroX-Tight-Pur-hSARS-Cov2 S wt or pRetroX-Tight-Pur-hSARS-Cov2 S Δ21 cells were stained with hoechst (1 ug/ml) or IHC stained using anti-SARS-CoV2-S antibody and viewed with a flourescence microscope.

FIG. 3 Immunogenicity evaluation of the cells expressing SARS-CoV2-S Δ21. a, b., ELISA test shows that the signal increases in accordance with the amount of cells expressing SARS-CoV2-S Δ21 (a) and the duration of dox administration (b) (hcPS (convalescent patient serum) was used as the primary antibody). C and d. ELISA test cells expressing SARS-CoV2-S wt, almost no signal was detected in Hep3B and Renca cells, respectively e. In the ELISA test using commercial anti-S1 antibody (Biolegend), the signal increases in accordance with the amount of cells expressing SARS-CoV2-S Δ21.

FIG. 4 SARS-CoV2-S Δ21 replaces the major cell membrane proteins including SPTA1 and CDH2

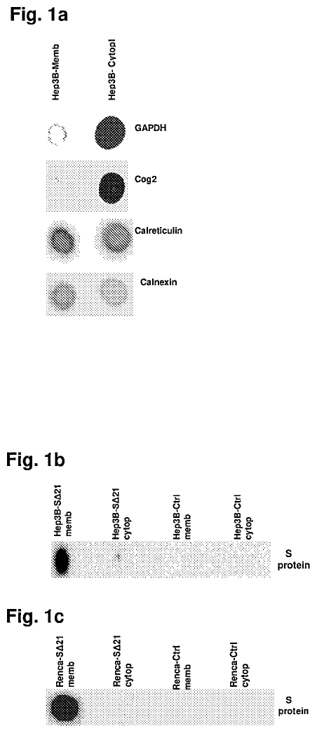

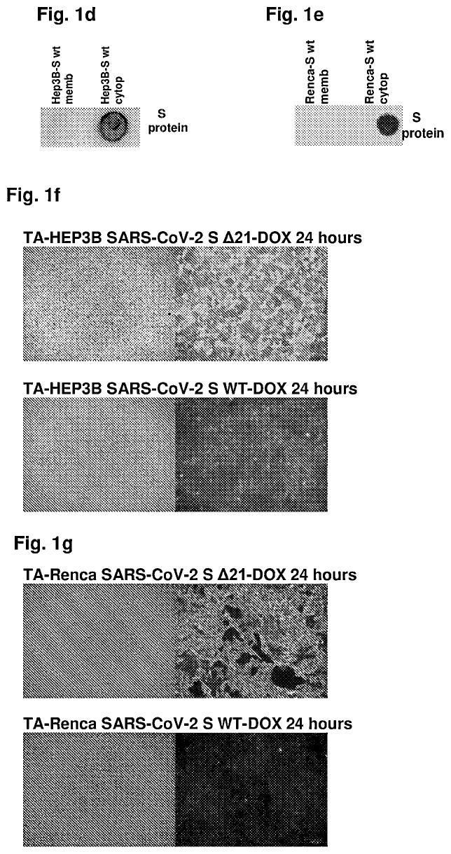

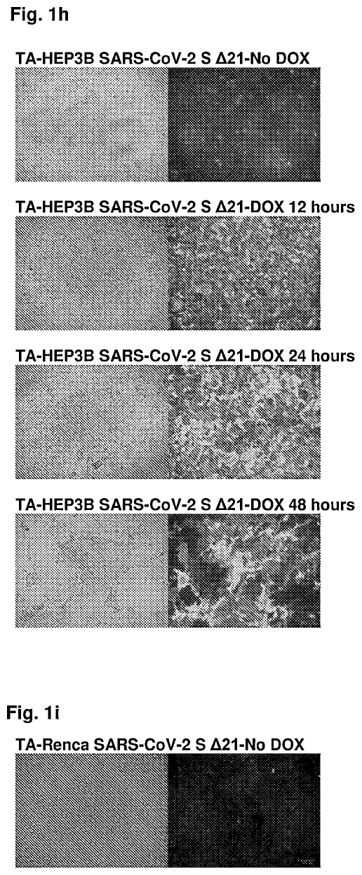

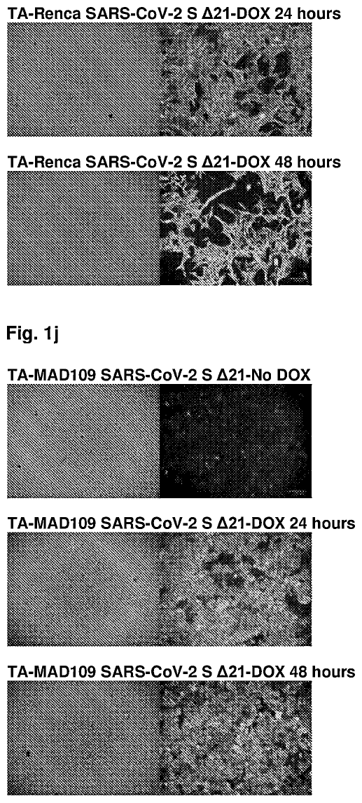

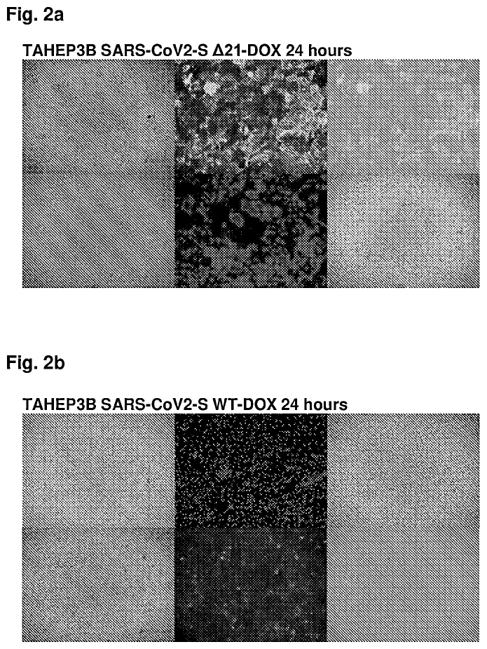

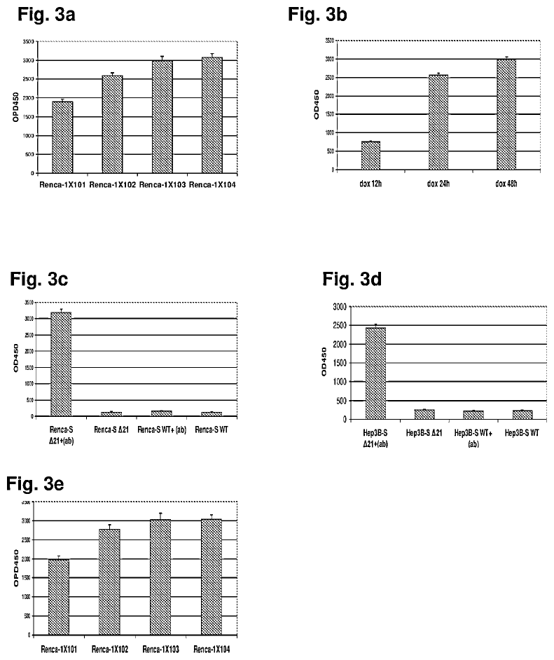

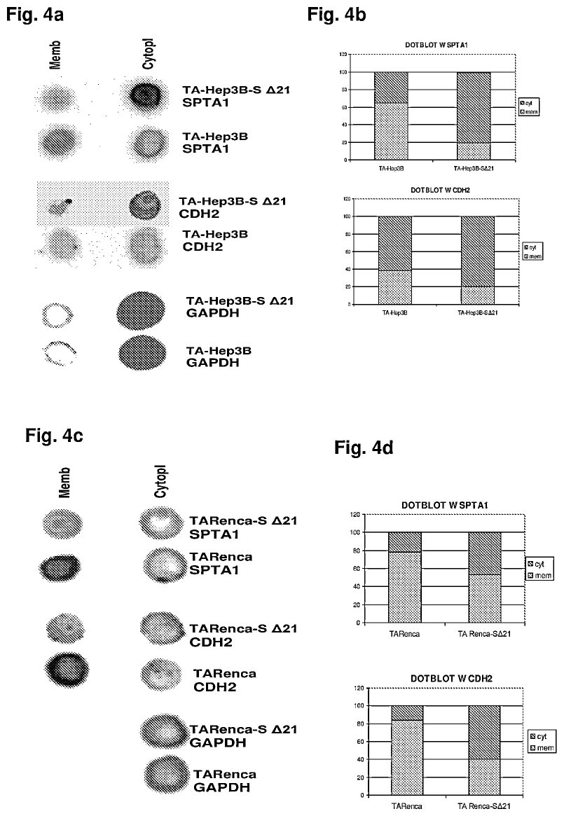

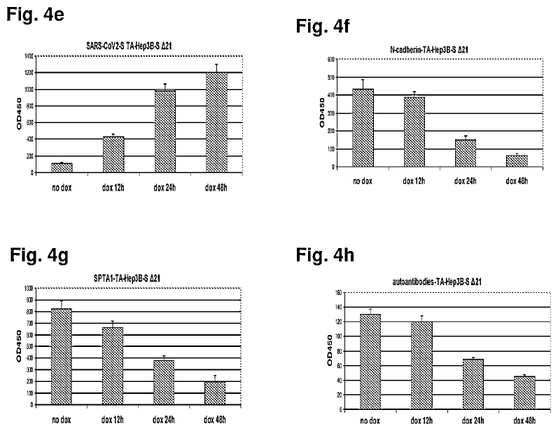

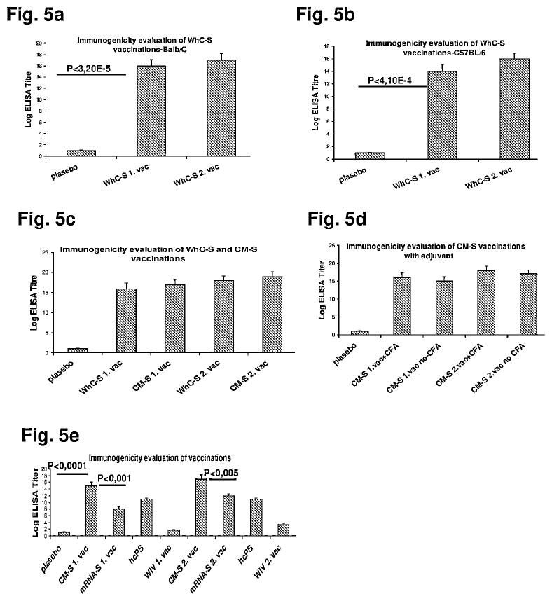

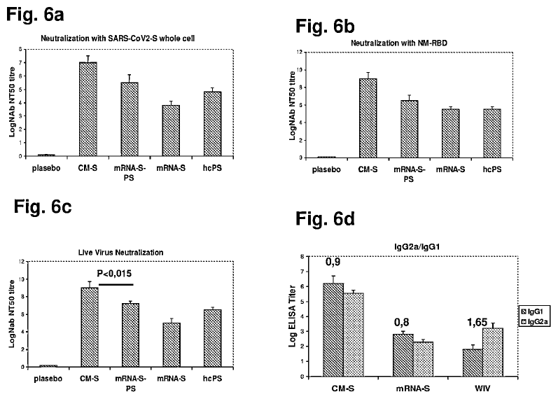

Cell membranes and cytoplasm of rtT expressing TA-Hep3B (control cell), and TA-Hep3B containing pRetroX-Tight-Pur-hSARS-CoV2-S Δ21 and TA-Renca (control cell), and TA-Renca containing pRetroX-Tight-Pur-mSARS-CoV2-S Δ21 were isolated after 36 hours of treatment with dox and the amount of SPTA1 and CDH2 proteins in cell membrane and cytoplasm of cells were investigated by dotblot using SPTA1 and CDH2 specific antibodies. a and b show dot blot and graphics of the abundance of SPTA1 and CDH2 proteins in the cytoplasm and membranes of TA-Hep3B (control) and TA-Hep3B-S Δ21 cells, respectively. c. and d. show dot blot and graphics of SPTA1 and CDH2 proteins in the cytoplasm and membranes of TA-Renca (control) and TA-Renca-S Δ21 cells, respectively. e-h. After dox addition, SARS-CoV2-S protein expression (e) and the amount of cell membrane proteins (f, g) and autoantibodies (h) (mSARS-CoV2-S negative human serum was used) were examined by ELISA at 0, 12, 24 and 48 hours in Renca cell containing pRetroX-Tight-Pur-mSARS-CoV2-S Δ21.

FIG. 5 Immunogenicity evaluation (IgG) of vaccinations with whole cell (WhC) or cell membrane (CM) obtained from the TA-Renca-mSARS-CoV2-S Δ21 in Balb/C mice (a), and C57BL/6 (b) (serums were taken 14 days after the 1^{st} vaccination and 14 days after the 2^{nd} vaccination with WhC-S) c. Results of vaccination with WhC-S were compared with the results of vaccination with CM-S. d. The effect of adding adjuvant to vaccination with CM-S. e. The result of immunogenicity evaluation of vaccination with CM-S was compared with the results of vaccination with mRNA-S, WIV (whole Inactivated Virus) and hcPS in Balb/C mice. h. Titer of neutralizing antibodies against live virus in the same groups.

FIG. 6. Protection efficacy of CM-S in the Balb/c mice against SARS-CoV-2. a. The neutralizing antibody titers of the the serums of CM-S, mRNA-S—PS, mRNA-S, and hcPS (Neutralization was completed with mSARS-CoV2-S Δ21 expressing Renca cell.). b. The neutralizing antibody titers of the the serums of CM-S, mRNA-S—PS (patient serum double immunized with mRNA-S), mRNA-S, and hcPS (Neutralization was completed with NM-RBD). c. Titer of neutralizing antibodies against live virus in the same groups. d. Titer ratios of IgG2a to IgG1 were in calculated in CM-S, mRNA-S, and WIV vaccinated Balb/c mice. Data are means t SEM (standard error of the mean). Comparisons were performed by Student's t-test (paired, two-tailed). Data are one representative result of three independent experiments.

### REFERENCES

Auld et al. (The assay Guidance Manual; https://www.ncbi.nlm.nih.gov/books/)
Boson B., Legros V., Zhou B., Siret E., Mathieu C., Cosset C. L., Lavillette D., and Denolly S. The SARS-CoV-2 envelope and membrane proteins modulate maturation and retention of the spike protein, allowing assembly of virus-like particles J. Biol. Chem. (2021) 296 100111
Brown T A (Genomes. 2nd edition. Oxford, Wiley-Liss:2002),
Cao H, Dan Z, He X, Zhang Z, Yu H, Yin Q, Li Y. Liposomes Coated with Isolated Macrophage Membrane Can Target Lung Metastasis of Breast Cancer. ACS Nano. 2016 Aug. 23; 10(8):7738-48.
Chiang C I I, Benencia F, and Coukos G Whole Tumor Antigen Vaccines Semin Immunol. 2010 June; 22(3): 132-143.
Golob J. L., Lugogo N. Lauring A. S. and Lok A. S. SARS-CoV-2 vaccines: a triumph of science and collaboration JCI Insight. 2021; 6(9):e149187.
Hotez P. J., and Bottazzi M. E. Whole Inactivated Virus and Protein-Based COVID-19 Vaccines Annual Review of Medicine; Sep. 27, 2021 15:35
Huang Y., Yang C., Xu X., Xu W., and Liu S. Structural and functional properties of SARS-CoV-2 spike protein: potential antivirus drug development for COVID-19 Acta Pharmacologica Sinica (2020) 41:1141-1149;
Liu Y, Luo J, Chen X, Liu W, Chen T. Cell Membrane Coating Technology: A Promising Strategy for Biomedical Applications. Nanomicro Lett. 2019 Nov. 16; 11(1):100.
Lontok E. Corse E. and Machamer C. E. Intracellular Targeting Signals Contribute to Localization of Coronavirus Spike Proteins near the Virus Assembly Site Journal of Virology, June 2004, p. 5913-5922 Vol. 78, No. 11
Miyanohara A. Preparation of vesicular stomatitis virus-G (VSV-G) conjugate and its use in gene transfer Cold Spring Harb Protoc. 2012 Apr. 1; 2012(4):453-6.
Molecular Cloning: A Laboratory Manual, 3nd Edition, Sambrook and Russel eds., Cold Spring Harbor Laboratory Press, Cold Spring Harbor 2001
Neller M A, Lópeza JA, Schmidt C W. Antigens for cancer immunotherapy Seminars in Immunology 20 (2008) 286-295
Nicolini A, Ferrari P, Biava P M. Exosomes and Cell Communication: From Tumour-Derived Exosomes and Their Role in Tumour Progression to the Use of Exosomal Cargo for Cancer Treatment. Cancers (Basel). 2021 Feb. 16; 13(4):822. doi: 10.3390/cancers13040822.
Radford K J, Tullett K M and Lahoud M H Dendritic cells and cancer immunotherapy. Curr Opin Immunol, 2014, 27, 26-32.
Rhea E M, Logsdon A F, Hansen K M, Williams L M, Reed M J, Baumann K K, Holden S J, Raber J, Banks W A, Erickson M A. The S1 protein of SARS-CoV-2 crosses the blood-brain barrier in mice. Nat Neurosci. 2021 March; 24(3):368-378. doi: 10.1038/s41593-020-00771-8.
Suzuki Y J, Nikolaienko S I, Dibrova V A, Dibrova Y V, Vasylyk V M, Novikov M Y, Shults N V, Gychka S G. SARS-CoV-2 spike protein-mediated cell signaling in lung vascular cells. Vascul Pharmacol. 2021 April; 137:106823. doi: 10.1016/j.vph.2020.106823.
Zhai Y, Su J, Ran W, Zhang P□, Yin Q, Zhang Z, Yu H, Li Y□ Preparation and Application of Cell Membrane-Camouflaged Nanoparticles for Cancer Therapy, Theranostics 2017, Vol. 7, Issue 10.

1-44. (canceled)

45. Use of a viral fusion glycoprotein for transporting an antigenic protein to the cell membrane and presenting it densely on the cell membrane.

46. The use of a viral fusion glycoprotein according to claim 45, wherein the viral fusion glycoprotein is selected from the group consisting of: the S2 subunit protein of SARS-CoV-2-S with the last 21 amino acids removed (SARS-CoV-2-S Δ21), the vesicular stomatitis virus glycoprotein (VSV-G), the influenza haemagglutinin (HA), the respiratory syncytial virus fusion glycoprotein (F), and human immunodeficiency virus gp160 (env) proteins.

47. The use of a viral fusion glycoprotein according to claim 45, wherein the viral fusion glycoprotein is the SARS-CoV-2-S Δ21, and wherein:
a) the FP, HR1, and HR2 segments are either removed or arranged differently;
b) the number of amino acids removed is less than 21 or more than 21 from the C-terminal end, and including mutations of any amino acid provided that such mutations maintain at least 30%, 40%, 50%, 60%, 70%, 80%, 85%, or 90% identity with the original sequence and retain the functionality of transporting the antigenic protein to the cell membrane.

48. A method for preparing a vaccine comprising:
a) Cloning the DNA sequence of a viral fusion glycoprotein according to claim 45 fused to the DNA sequence of an antigenic protein of interest into an expression system;
b) Transfecting or infecting target cells with said expression system;
c) Inducing the expression of the SARS-CoV-2-S Δ21 protein fused to the antigenic protein in the cells of step b);
d) Isolating the cells or cell membranes containing the antigenic protein (membrane-antigenic protein complex).

49. The method according to claim 48, wherein the expression system is selected from the group consisting of plasmids, retroviruses, lentiviruses, adenoviruses, adeno-associated viruses (AAV), bacteriophages, or any other viral or non-viral vector systems.

50. The method according to claim 48, wherein the expression system is a tetracycline-inducible mammalian expression system and, more preferably, the Retro-X Tet-On Advanced Inducible Expression System.

51. The method according to claim 48, wherein target cells are selected from the group consisting of primary, secondary, immortalized, or stem cell lines.

52. A cell or cell membrane (membrane-antigenic protein complex) obtainable by the method according claim 48.

53. The viral fusion glycoprotein according to claim 45, wherein the antigenic protein of interest is selected from the group consisting of viral proteins, bacterial proteins, tumor-associated proteins, and specific antigenic proteins derived from pathogens.

54. The viral fusion glycoprotein, the method, or the cell or cell membrane (membrane-antigenic protein complex) according to claim 53, wherein the antigenic protein of interest is selected from the group consisting of spike proteins and receptor binding domain (RBD) proteins of SARS-CoV-2, preferably the S1 subunit.

55. A composition comprising the viral fusion glycoprotein or the cell or cell membrane (membrane-antigenic protein complex) according to claim 45, for use as a medicament, preferably a vaccine.

56. The composition for use according to claim 55, wherein the composition is administered to a subject, and wherein:
a) The dose ranges from 10³ to 10¹⁰ cells or cell membranes per dose, preferably 10³ to 10⁹, and particularly 10⁴ to 10⁶;
b) A subject usually receives a single injection, with additional injections administered at weekly or monthly intervals as needed until treatment efficacy is established, determined by evaluating symptoms, clinical parameters, or the desired immunological response;
c) The composition is delivered subdermally, intradermally, or intramuscularly, with the best route depending on factors such as the species, age, gender, overall condition of the subject, and the nature and severity of the condition being treated, as well as the specific composition being administered.

57. The use of a viral fusion glycoprotein, the cell or cell membrane (membrane-antigenic protein complex), or the composition according to claim 55, for antibody production.

58. The antibody according to claim 57, for use in therapy.

59. The use of a viral fusion glycoprotein, the cell or cell membrane (membrane-antigenic protein complex), the composition according to claim 55, for the development of an immunodiagnostic test kit.
